(19) United States
(12) Patent Application Publication
(10) Pub. No.: US 2019/0314382 A1
(43) Pub. Date: Oct. 17, 2019

(54) COMPOUNDS AND PHARMACEUTICAL COMPOSITIONS THEREOF FOR THE TREATMENT OF INFLAMMATORY DISEASES

(71) Applicant: Galapagos NV, Mechelen (BE)

(72) Inventors: Chantal Thérèse TASSET, Mechelen (BE); Pille HARRISON, Juan les Pins (FR); René Alexandre GALIEN, Romainville (FR); John Sargent SUNDY, Foster City, CA (US); John G. MCHUTCHISON, Foster City, CA (US); Thomas O'RIORDAN, Norwell, MA (US); Neelufar MOZAFFARIAN, Mercer Island, WA (US); Uptal Dinesh PATEL, Foster City, CA (US); Timothy R. WATKINS, Foster City, CA (US); David L. GOSSAGE, Hingham, MA (US)

(21) Appl. No.: 16/348,799

(22) PCT Filed: Nov. 9, 2017
(86) PCT No.: PCT/EP2017/078701

§ 371 Date: May 9, 2019

(30) Foreign Application Priority Data
Nov. 10, 2016 (GB) 1618959.9
Nov. 10, 2016 (GB) 1618960.7
Nov. 10, 2016 (GB) 1618961.5
Nov. 10, 2016 (GB) 1618962.3
Nov. 10, 2016 (GB) 1618963.1
Nov. 10, 2016 (GB) 1618964.9

(51) Int. Cl. A61K 31/541; A61K 45/06; A61P 13/12; A61P 17/14

(52) U.S. Cl. CPC A61K 31/541; A61P 17/14; A61P 13/12; A61K 45/06

(57) ABSTRACT

The present invention relates to the use of compounds or pharmaceutical compositions comprising the same, and methods of treatment using the same, in the prophylaxis and/or treatment of alopecia areata, vitiligo, cutaneous lupus, lupus nephritis, giant cell arteritis, sarcoïdosis and/or sarcoïdosis-related conditions. In particular the compounds are JAK inhibitors.

### FIELD OF THE INVENTION

The present invention relates to compounds useful in the prophylaxis and/or treatment of alopecia areata, vitiligo, cutaneous lupus, lupus nephritis, giant cell arteritis, sarcoïdosis, and/or a sarcoïdosis-related condition. In particular, the compounds of the invention inhibit JAK. More particularly, the compounds of the invention inhibit JAK1. The present invention also provides methods for the prophylaxis and/or treatment of alopecia areata, vitiligo, cutaneous lupus, lupus nephritis, giant cell arteritis, sarcoïdosis, and/or a sarcoïdosis-related condition comprising administering a compound of the invention.

### BACKGROUND OF THE INVENTION

Alopecia areata is an autoimmune disease resulting in hair loss with devastating psychosocial consequences. Despite its high prevalence, affecting millions of people worldwide, in the United States, alopecia areata was estimated to occur in 0.1% to 0.2% of the general population, with a lifetime risk of 1.7% (Alkhalifah et al., 2010).

It manifests as a sudden loss of hair without associated visible scarring or inflammation of the skin. In 5% of patients affected by alopecia areata, the condition can extend to involve the entire scalp (alopecia totalis (AT)) or the whole body (alopecia universalis; AU). Alopecia areata can involve nail changes, increased atopy, and other associated autoimmune and endocrine disorders, such as thyroid disease.

Alopecia areata rates are similar in both men and women and the majority of patients are between the ages of 30-59 (Wang and McElwee, 2011). Alopecia areata tends to worsen with time in children and be more persistent (Alkhalifah et al., 2010). High rates of anxiety and major depression have been shown to be co-morbid with alopecia areata. (Karia et al., 2015) The prognosis of alopecia areata is worse in patients who have AT or AU, an ophiasis pattern of hair loss, a family history of the condition, disease onset at a younger age, nail involvement, and other coexisting autoimmune diseases (Alkhalifah et al., 2010).

To date, no treatments for alopecia areata have been approved by the FDA. Nevertheless, in attempting to address this disease, prior studies have identified a prominent interferon gamma (IFN-γ) signature in alopecia areata, which signals through JAK. (Xing et al., 2014).

Although the psychosocial stress resulting from the disease may be traumatizing to the patient and cause of morbidity (Karia et al., 2015), alopecia areata alone is not a life threatening condition, and accordingly a suitable treatment agent should be safe, with low associated side effects.

Vitiligo is an autoimmune disease where depigmentation of the skin occurs thus resulting in disfiguring white spots which negatively impact on the patient self-esteem (Allam and Riad, 2013). Vitiligo occurs worldwide with an estimated prevalence of 0.5-1% in most populations. In almost half of patients, vitiligo starts before the age of 20 years, and males and females are affected with approximately equal frequency. (Ezzedine et al., 2012)

Although treatments to attenuate the disease exist, ranging from topical steroid application and/or calcineurin inhibitors to phototherapy (Allam and Riad, 2013), to date no treatments for vitiligo have been approved by the FDA. Nevertheless, in attempting to address this disease, prior studies have identified a prominent interferon gamma (IFN-γ) signature including the chemokine CXCL9, 10 and 11 (Rashighi et al., 2014). When IFN-γ binds to its receptor, it activates the JAK/STAT pathway, which in turn leads to production of CXCL10 in keratinocytes. Therefore, a direct CXCL10 inhibition or a JAK/STAT pathway inhibition may represent attractive targets for new treatment strategies (Di Lernia and Bardazzi, 2016).

Although the psychosocial stress resulting from the disease may be traumatizing to the patient and be a contributor to morbidity (Karia et al., 2015), vitiligo alone is not a life threatening condition, and accordingly a suitable treatment agent should be safe, with low associated side effects.

Lupus or lupus erythematosus is an autoimmune disorder that can cause chronic inflammation in various parts of the body, especially the skin, joints, blood, and kidneys. The three most common types of lupus include systemic lupus erythematosus (SLE), cutaneous lupus erythematosus (CLE) and drug-induced lupus.

Cutaneous lupus erythematosus (CLE) or cutaneous lupus (CL) affects primarily the skin and is generally characterized by skin inflammation, skin rashes and hemorrhages in the skin. This inflammation is driven by interferon (IFN)-regulated pro-inflammatory cytokines regulating the B- and T-cell mediated lesional inflammation (Robinson and Werth, 2015). Cutaneous lupus may also affect hair and mucous membranes, but usually does not involve internal organs like SLE. Cutaneous lupus can be categorized into groups including acute cutaneous lupus erythematosus (ACLE), subacute cutaneous lupus erythematosus (SCLE), and chronic cutaneous lupus erythematosus (CCLE).

ACLE is often seen in patients with active systemic disease. Flat red patches are seen on the cheeks and nose and are referred to as a butterfly rash, these are very common in ACLE. Individuals may also have flat red patches on arms, legs and trunk. These lesions are sensitive to UV light and commonly appear on sun-exposed areas.

SCLE is a non-scarring non-atrophy-producing photosensitive dermatosis. In some cases, SCLE appears as a non-itchy ring-shaped dry rash on the upper back and chest, often following sun exposure. SCLE may occur in patients with systemic lupus erythematosus (SLE), Sjogren's syndrome and deficiency of the second component of complement (C2d). Sometimes it can be also drug-induced.

CCLE or discoid lupus erythematosus (DLE) is a chronic, scarring, atrophy producing, photosensitive dermatosis. DLE commonly appears as red scaly patches which leave white scars. DLE predominantly affects the cheeks and nose, but sometimes involves the upper back, neck, backs of hands, bald areas on the scalp, and the lips. DLE may occur in patients with SLE.

There have been numerous studies to understand the underlying mechanism of the disease and identify potential target for treatments (Kuhn et al., 2006).

CLE gene expression analyses revealed lesional activation of several pathways of the immune system, thus providing potential new therapeutic targets. There are indications of the involvement of specific pathways and targets in the pathogenesis of cutaneous lupus erythematosus, including TLR-dependent and TLR-independent immune activation, NfkB, TBK1, PI3K, MAPK, and JAK/STAT-pathway. (Klaeschen and Wenzel, 2016)

In treating CLE, dermatologists aim to prevent the formation and progression of lesions, and to improve skin appearance through a combination of patient education, and topical and systemic therapies. Treatment of CLE lesions currently begins with topical therapies, including steroids and/or calcineurin inhibitors. Systemic therapies are indicated in cases where there is widespread or scarring disease, or in cases refractory to topical treatments. When systemic treatments are prescribed, topical agents are typically continued as adjunctive therapy. Presently, there are no medications specifically approved for the treatment of CLE. The drugs used for the treatment of the various subtypes of CLE are generally also used for the treatment of SLE, with the exception of thalidomide.

However, these drugs are not always effective for treating some cutaneous lupus and/or treat only general symptoms of CLE and/or they may have serious side effects when they are continuously used for a long period of time.

Lupus nephritis (LN) is an inflammation of the kidneys caused by systemic lupus erythematosus (SLE). Lupus nephritis is staged according to the classification revised by the International Society of Nephrology (ISN) and the Renal Pathology Society (RPS) in 2003 into several classes (Classes I-VI) (Lewis et al., 2010).

Approximately 60% of patients with systemic lupus erythematosus develop kidney disease (Cameron et al, 2001, Cameron J S: Systemic lupus erythematosus. In: Immunologic Renal Diseases, 2nd Ed., edited by Neilson E G, Couser W G, Philadelphia, Lippincott-Raven, 2001, pp 1057-1104). Although most have proliferative (class II, III, or IV) lupus nephritis, a subgroup of patients develop pure Membranous Lupus Nephritis (MLN; class V). In contrast to class III or IV lupus nephritis, MLN may present without other clinical or serologic manifestations of lupus. MLN is characterized by diffuse thickening of the glomerular capillary wall (segmentally or globally), with diffuse membrane thickening, and subepithelial deposits visible under the electron microscope. Clinically, MLN presents with signs of nephrotic syndrome

Although renal pathology is highly predictive of the disease course in lupus nephritis, it cannot be performed serially because of its invasive nature and associated morbidity. One study (Singh et al., 2012) found elevated urinary levels of CXC ligand 16 (CXCL16), monocyte chemotactic protein-1 (MCP-1) and vascular cell adhesion molecule-1 (VCAM-1) in patients with lupus nephritis.

Membranous lupus nephritis is characterized by formation of immune complexes deposits which can induce an inflammatory response by activation of adhesion molecules on endothelium, resulting in the recruitment of pro inflammatory leukocytes Immune cells produce cytokines that play a pivotal role as inflammatory mediators to extend renal injury. In serum of SLE patients, the concentrations of IL-6, IL-17, IL-12, IFNγ, IL-18, IL-10 and TNF-alpha are higher than healthy people and this increase correlate with disease activity. There is a well established possible correlation between urinary cytokines levels (IL-6, IL-10, IFNγ and TGFβ) and disease activity (Gigante et al., 2011)

While little is known about inflammatory cytokine activation in lupus nephritis specifically, one recent study (Wang et al., 2010) attempted to evaluate the role of the cytokines in lupus nephritis and the role of JAK/STAT (Janus kinase-signal transducer and activator of transcription factor), which pathway is important in mediating signal transduction of cytokines. JAK2 inhibitor tyrphostin AG490 treatment of MRL/lpr mice significantly inhibited the renal expression of monocyte chemotactic protein (MCP)-1, interferon (IFN)-γ and class II MHC, which was accompanied by reduced renal infiltration of T cells and macrophages. This implies that JAK/STAT pathway is implicated in the progression of renal inflammation in MRL/lpr mice and targeting this pathway may provide a potential therapeutic approach for lupus nephritis.

There is no definitive treatment or cure for lupus nephritis. Commonly used therapy normalizes renal function, urine sediment and proteinuria, reduces the frequency of relapses or prevents the progressive loss of renal function through mild, moderate and severe renal impairment to end stage renal disease (ESRD) requiring dialysis or kidney transplantation. Therapy varies pending on the histopathological findings as well as the clinical manifestations.

Treatment of LN largely involves immunosuppressive agents, and schedules that prove effective in both idiopathic membranous nephropathy and proliferative lupus nephritis often include alkylating agents and calcineurin inhibitors. However, because of the varied agents and regimens and the relatively few patients with MLN who compose most clinical trials, there is no consensus as to the most effective treatment for membranous disease (Kolasinski et al., 2002).

Some investigational therapies for lupus nephritis include anti CD20 monoclonal antibodies, anti-cytokine therapies against IFNα, IL1, IL6, and TNFα, or with IL10-Fc protein.

Sarcoïdosis, also called sarcoid disease or Besnier-Boeck disease, is a multisystem inflammatory disease that can affect a variety of organs (e.g., lungs, skin, heart, eyes, liver, nervous system, kidneys). The etiology of sarcoïdosis remains unknown. It has been linked to alterations in the immune response after exposure to an environmental or infectious agent in susceptible individuals.

Sarcoïdosis is a disease characterized by the growth of tiny collections of inflammatory cells (granulomas) in different parts of the body. Such granulomas are caused by a disproportionate Th1 granulomatous immune response in the organs involved (Iannuzzi et al., 2007). They are commonly found in the lungs, lymph nodes, eyes and skin. Th1 lymphocytes predominantly secrete interleukin-2 and interferon gamma, enhance macrophage tumor necrosis factor (TNF) alpha production and amplify the local cellular immune response. A variety of exogenous agents, both infectious and non-infectious, have been proposed to trigger disproportionate immune response (Baughman et al., 2003).

Sarcoïdosis may be acute or chronic. Specific subtypes of sarcoïdosis include, but are not limited to, cardiac sarcoïdosis, cutaneous sarcoïdosis, hepatic sarcoïdosis, oral sarcoïdosis, pulmonary sarcoïdosis, neurosarcoïdosis, sinonasal sarcoïdosis, Lofgren's syndrome, and chronic cutaneous sarcoïdosis

Rosenbaum et al. demonstrated that RNA for the major transcription factor, STAT1, is upregulated in the peripheral blood of patients with sarcoïdosis compared to healthy controls. (Rosenbaum et al., 2009) In addition, mRNAs for 13 of the 18 genes directly regulated by STAT1 have a statistically significant increase in the blood of patients with sarcoïdosis. The STATs are activated by Janus protein tyrosine kinases (JAKs). Although signaling through many receptors is dependent on JAK-STAT activation, interferons are especially dependent on this pathway. The study also demonstrated that CXCL9, STAT1 and Pim-1 were upregulated in sarcoïdosis patients.

Presently there is no cure for sarcoïdosis. Antibiotics are being commonly used for the treatment of sarcoïdosis, such as penicillin antibiotics, cephalosporin antibiotics, macrolide antibiotics, lincomycin antibiotics, and tetracycline antibiotics (see WO 2005/002623 (“Remedy for Sarcoidosis and Method of Treating the Same,” 2006)).

Corticosteroid drugs remain the primary treatment for the inflammation and granuloma formation associated with sarcoïdosis. (Rizzato et al., 1997). However, the use of corticosteroids has a number of drawbacks. For example, certain patients do not respond to steroid therapy. In addition, corticosteroids have several serious side effects, and their use is typically limited to progressive or severe conditions.

Accordingly, there exists a need for new methods of treating sarcoïdosis. The present invention provides the compound of the invention for use in the prophylaxis and/or treatment of sarcoïdosis and/or a sarcoïdosis-related condition.

Giant cell arteritis (GCA) also known as temporal arteritis or cranial arteritis or Horton disease is the most common form of primary systemic vasculitis which is an inflammatory disease of blood vessels predominantly involving large and medium arteries of the head, and more particularly the branches of the external carotid artery. (Ponte et al., 2015; Rutgers et al., 2016)

The incidence rate of GCA has been reported to range between 15 to 25 individuals in 100,000 per annum (Ponte et al., 2015). Early GCA symptoms include constitutional symptoms, headache, visual loss, polymyalgia rheumatica (PMR), and jaw claudication, and may lead to blindness in up to 20% of GCA afflicted individuals. Glucocorticoids are the cornerstone of medical therapy in GCA, however they lead to significant toxicity in over 80% of the patients. For example, a high number of major adverse advents related to long-term glucocorticoid use in GCA have been reported: posterior subcapsular cataract (41%), bone fractures (38%), infections (31%), hypertension (22%), diabetes mellitus (9%) and gastrointestinal bleeding (4%). (Proven et al., 2003)

Monitoring of disease progress still relies on assessing clinical features and measuring inflammatory markers (C-reactive protein and erythrocyte sedimentation rate). (Ponte et al., 2015)

Recently, it has been observed that patients with GCA, refractory to glucocorticoids, have a higher expression of pro-inflammatory cytokines such as IL-1β, TNF-α, and IL-6. Moreover, mice lacking the interleukin 1 receptor antagonist (IL-1ra) gene developed large vessel vasculitis, suggesting that IL-1 inhibition could be a therapeutic option in patients with GCA. (Shepherd and Nicklin, 2005)

Monoclonal IL-6 blockers are under evaluation, but clearly available treatment agents are lacking.

In fighting RA, Janus kinase (JAK) inhibitors have been developed. JAKs are cytoplasmic tyrosine kinases that transduce cytokine signaling from membrane receptors to STAT transcription factors. Four JAK family members have been described, JAK1, JAK2, JAK3 and TYK2. Upon binding of the cytokine to its receptor, JAK family members auto- and/or transphosphorylate each other, followed by phosphorylation of STATs that then migrate to the nucleus to modulate transcription. JAK-STAT intracellular signal transduction serves the interferons, most interleukins, as well as a variety of cytokines and endocrine factors such as EPO, TPO, GH, OSM, LIF, CNTF, GM-CSF and PRL (Vainchenker et al., 2008).

The combination of genetic models and small molecule JAK inhibitor research revealed the therapeutic potential of inhibition of several JAKs.

JAK1 is a target in the immuno-inflammatory disease area. JAK1 heterodimerizes with the other JAKs to transduce cytokine-driven pro-inflammatory signaling. Therefore, inhibition of JAK1 is of interest for immuno-inflammatory diseases with pathology-associated cytokines that use JAK1 signaling, such as IL-6, IL-4, IL-9, IL-15, IL-21, or IFNγ, as well as for other diseases driven by JAK-mediated signal transduction. The compound according to Formula I, cyclopropanecarboxylic acid {5-[4-(1,1-dioxo-thiomorpholin-4-ylmethyl)-phenyl]-[1,2,4]triazolo[1,5-a]pyridin-2yl}-amide (Compound 1), is disclosed in WO2010/149769 (Menet and Smits, 2010) and has the chemical structure shown below:

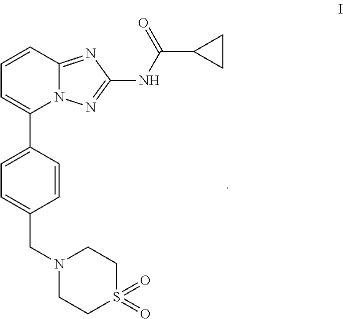 [SMILES: O=C(Nc1nc2cccc(-c3ccc(CN4CCS(=O)(=O)CC4)cc3)n2n1)C1CC1]

Compound 1 is a JAK inhibitor, more particularly a JAK1 inhibitor, and useful in the treatment of inflammatory conditions, autoimmune diseases, proliferative diseases, allergy, transplant rejection, diseases involving impairment of cartilage turnover, congenital cartilage malformations, and/or diseases associated with hypersecretion of IL6 or interferons.

However, whereas JAK inhibitors are useful and effective molecules in the treatment of RA, or inflammatory bowel disorders (IBD) including Crohn's disease, drawbacks to the use of these compounds have been reported including anemia, thrombocytopenia and neutropenia, hypercholesterolemia, creatinine increase, all of which may result from the lack of selectivity, in particular selectivity against JAK2 (O'Shea et al., 2013; O'Shea and Plenge, 2012). In contrast, selective JAK inhibition, in particular JAK1 may result in safe and effective treatment agent. (Yamaoka, 2016)

Accordingly, there is a need for molecules that may be used for the prophylaxis and/or treatment of alopecia areata, and which are safe with low associated side effects.

Therefore, there is a need to identify compounds and develop new therapeutic methods of treating cutaneous lupus and its different forms.

There is, therefore also, a need for alternative therapies with better risk-benefit profiles for the treatment of lupus nephritis.

Accordingly, there is a need for molecules for the prophylaxis and/or treatment of giant cell arteritis which are safe with low associated side effects. Accordingly, there exists a need for new methods of treating sarcoïdosis.

### SUMMARY OF THE INVENTION

The present invention provides the compounds of the invention, e.g. a compound according to Formula I (Compound 1), for use in the prophylaxis and/or treatment of alopecia areata. In particular, the compounds of the invention may act as inhibitors of JAK, and more particularly of JAK1.

Furthermore, the present invention provides pharmaceutical compositions comprising a compound of the invention, e.g. a compound according to Formula I (Compound 1), for use in the prophylaxis and/or treatment of alopecia areata.

The present invention also provides methods for the production of these pharmaceutical compositions of the invention and methods for the prophylaxis and/or treatment of alopecia areata by administering the pharmaceutical compositions of the invention.

The present invention provides the compounds of the invention, e.g. a compound according to Formula I (Compound 1), for use in the prophylaxis and/or treatment of vitiligo. In particular, the compounds of the invention may act as inhibitors of JAK, and more particularly of JAK1.

Furthermore, the present invention provides pharmaceutical compositions comprising a compound of the invention, e.g. a compound according to Formula I (Compound 1), for use in the prophylaxis and/or treatment of vitiligo.

The present invention also provides methods for the production of these pharmaceutical compositions of the invention and methods for the prophylaxis and/or treatment of vitiligo by administering the pharmaceutical compositions of the invention.

The present invention provides the compounds of the invention, e.g. a compound according to Formula I (Compound 1) for use in the prophylaxis and/or treatment of cutaneous lupus. In particular, the compounds of the invention may act as inhibitors of JAK, and more particularly of JAK1.

Furthermore, the present invention provides pharmaceutical compositions comprising a compound of the invention, e.g. a compound according to Formula I (Compound 1), for use in the prophylaxis and/or treatment of cutaneous lupus.

The present invention also provides methods for the production of these pharmaceutical compositions of the invention and methods for the prophylaxis and/or treatment of cutaneous lupus by administering the pharmaceutical compositions of the invention.

The present invention provides a compound of the invention, e.g. a compound according to Formula I (Compound 1) for use in the prophylaxis and/or treatment of lupus nephritis. In particular, the compounds of the invention may act as inhibitors of JAK, and more particularly of JAK1.

Furthermore, the present invention provides pharmaceutical compositions comprising a compound of the invention, e.g. a compound according to Formula I (Compound 1), for use in the prophylaxis and/or treatment of lupus nephritis.

The present invention also provides methods for the production of these pharmaceutical compositions of the invention and methods for the prophylaxis and/or treatment of lupus nephritis by administering the pharmaceutical compositions of the invention.

The present invention provides a compound of the invention, e.g. a compound according to Formula I (Compound 1) for use in the prophylaxis and/or treatment of sarcoïdosis and/or a sarcoïdosis-related condition. In particular, the compounds of the invention may act as inhibitors of JAK, and more particularly of JAK1.

Furthermore, the present invention provides pharmaceutical compositions comprising a compound of the invention, e.g. a compound according to Formula I (Compound 1), for use in the prophylaxis and/or treatment of sarcoïdosis and/or a sarcoïdosis-related condition.

The present invention also provides methods for the production of these pharmaceutical compositions of the invention and methods for the prophylaxis and/or treatment of sarcoïdosis and/or a sarcoïdosis-related condition by administering the pharmaceutical compositions of the invention.

The compound according to Formula I, a JAK1 selective inhibitor, has shown remarkable efficacy in phase II for the prophylaxis and/or treatment of rheumatoid arthritis, and inflammatory bowel diseases, in particular Crohn's disease, where it has demonstrated a remarkable safety profile, in particular regarding anemia, lymphocytes, neutrophils, hemoglobin (WO 2016/165953 (Wigerinck et al., 2016)), and also showed a remarkable improvement in lipid profile in patients (WO 2016/165952 (Wigerinck and Van't Klooster, 2016)).

The compounds of the invention may be of use in the prevention and/or treatment of alopecia areata, and therefore the present invention provides the compounds of the invention for use in the prophylaxis and/or treatment of alopecia areata.

The compounds of the invention may be of use in the prevention and/or treatment of vitiligo, and therefore the present invention provides the compounds of the invention for use in the prophylaxis and/or treatment of vitiligo.

The compounds of the invention may be of use in the prevention and/or treatment of cutaneous lupus, and therefore the present invention provides the compounds of the invention for use in the prophylaxis and/or treatment of cutaneous lupus.

The compound of the invention may be of use in the prevention and/or treatment of cutaneous lupus, and therefore the present invention provides the compounds of the invention for use in the prophylaxis and/or treatment of lupus nephritis.

The compounds of the invention may be of use in the prevention and/or treatment of giant cell arteritis, and therefore the present invention provides compounds of the invention for use in the prophylaxis and/or treatment of sarcoïdosis and/or a sarcoïdosis-related condition.

The compounds of the invention may be of use in the prevention and/or treatment of giant cell arteritis, and therefore the present invention provides compounds of the invention for use in the prophylaxis and/or treatment of giant cell arteritis.

Accordingly, in a first aspect of the invention, the compound of the invention is provided according to Formula (I):

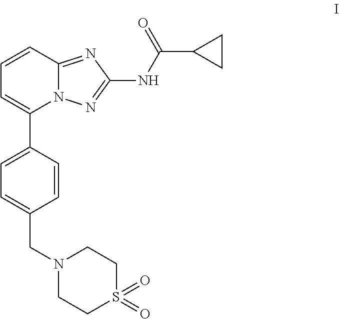 [SMILES: O=C(Nc1nc2cccc(-c3ccc(CN4CCS(=O)(=O)CC4)cc3)n2n1)C1CC1]

for use in the prophylaxis and/or treatment of alopecia areata.

The present invention also provides pharmaceutical compositions comprising a compound of the invention, and a suitable pharmaceutical carrier, excipient or diluent for use in the prophylaxis and/or treatment of alopecia areata.

In a further particular aspect, the pharmaceutical compositions may additionally comprise further therapeutically active ingredients suitable for use in combination with the compounds of the invention. In a more particular aspect, the further therapeutically active ingredient is an agent for the treatment of alopecia areata.

In one aspect of the invention, this invention provides a method for the prophylaxis and/or treatment of alopecia areata in a mammal in need thereof, in particular humans, which method comprises administering an effective amount of a pharmaceutical composition or compound of the invention as described herein.

In one aspect, this invention provides the compounds of the invention for use in the prophylaxis and/or treatment of alopecia areata in a mammal, in particular humans, afflicted with alopecia areata. In a particular embodiment, the presence of alopecia areata is determined using the SALT score. In a specific embodiment, the presence of alopecia areata is defined by a SALT score of at least 25%, at least 50%, at least 75%, or 100%.

The present invention also provides pharmaceutical compositions comprising the compounds of the invention, and a suitable pharmaceutical carrier, excipient or diluent for use in medicine. In a particular aspect, the pharmaceutical composition is for use in the prophylaxis and/or treatment of alopecia areata.

Accordingly, in a second aspect of the invention, the compound of the invention is provided according to Formula (I) for use in the prophylaxis and/or treatment of vitiligo.

The present invention also provides pharmaceutical compositions comprising a compound of the invention, and a suitable pharmaceutical carrier, excipient or diluent for use in the prophylaxis and/or treatment of vitiligo.

In a further particular aspect, the pharmaceutical compositions may additionally comprise further therapeutically active ingredients suitable for use in combination with the compounds of the invention. In a more particular aspect, the further therapeutically active ingredient is an agent for the treatment of vitiligo.

Moreover, the compounds of the invention, useful in the pharmaceutical compositions and treatment methods disclosed herein, are pharmaceutically acceptable as prepared and used.

In one aspect of the invention, this invention provides a method for the prophylaxis and/or treatment of vitiligo in a mammal in need thereof, in particular humans, which method comprises administering an effective amount of a pharmaceutical composition or compound of the invention as described herein.

In one aspect, this invention provides the compounds of the invention for use in the prophylaxis and/or treatment of vitiligo in a mammal, in particular humans. In a particular embodiment, the presence of vitiligo is determined using the VASI score. In a specific embodiment, the presence of vitiligo is defined by a VASI score of at least 10%, at least 25%, at least 50%, at least 75%, at least 90%, or 100%.

The present invention also provides pharmaceutical compositions comprising a compound of the invention, and a suitable pharmaceutical carrier, excipient or diluent for use in medicine. In a particular aspect, the pharmaceutical composition is for use in the prophylaxis and/or treatment of vitiligo.

Accordingly, in a third aspect of the invention, the compound of the invention is provided according to Formula (I), for use in the prophylaxis and/or treatment of cutaneous lupus.

The present invention also provides pharmaceutical compositions comprising a compound of the invention, and a suitable pharmaceutical carrier, excipient or diluent for use in the prophylaxis and/or treatment of cutaneous lupus.

In a further particular aspect, the pharmaceutical composition may additionally comprise further therapeutically active ingredients suitable for use in combination with the compounds of the invention. In a more particular aspect, the further therapeutically active ingredient is an agent for the treatment of cutaneous lupus.

In one aspect of the invention, this invention provides a method for the prophylaxis and/or treatment of cutaneous lupus in a mammal in need thereof, in particular humans, which method comprises administering an effective amount of a pharmaceutical composition or compound of the invention as described herein.

In one aspect, this invention provides the compound of the invention for use in the prophylaxis and/or treatment of cutaneous lupus in a mammal, in particular humans. In a more specific embodiment the severity of cutaneous lupus is determined using the Cutaneous Lupus Erythematosus Disease Area and Severity Index (CLASI). In a particular embodiment severe CL is determined by a CLASI score of at least 21.

The present invention also provides pharmaceutical compositions comprising a compound of the invention, and a suitable pharmaceutical carrier, excipient or diluent for use in medicine. In a particular aspect, the pharmaceutical composition is for use in the prophylaxis and/or treatment of cutaneous lupus.

Accordingly, in a first aspect of the invention, the compound of the invention is provided having a Formula (I), for use in the prophylaxis and/or treatment of lupus nephritis.

The present invention also provides pharmaceutical compositions comprising a compound of the invention, and a suitable pharmaceutical carrier, excipient or diluent for use in the prophylaxis and/or treatment of lupus nephritis.

In a further particular aspect, the pharmaceutical composition may additionally comprise further therapeutically active ingredients suitable for use in combination with the compounds of the invention. In a more particular aspect, the further therapeutically active ingredient is an agent for the treatment of lupus nephritis.

In one aspect of the invention, this invention provides a method for the prophylaxis and/or treatment of lupus nephritis in a mammal in need thereof, in particular humans, which method comprises administering an effective amount of a pharmaceutical composition or compound of the invention as described herein.

In one aspect, this invention provides the compounds of the invention for use in the prophylaxis and/or treatment of lupus nephritis in a mammal, in particular humans, afflicted with lupus nephritis. In a more particular embodiment the effect of the treatment is measured by proteinuria reduction by 24-hour urine protein level.

The present invention also provides pharmaceutical compositions comprising a compound of the invention, and a suitable pharmaceutical carrier, excipient or diluent for use in medicine. In a particular aspect, the pharmaceutical composition is for use in the prophylaxis and/or treatment of lupus nephritis.

Accordingly, in a first aspect of the invention, a compound of the invention is provided having a Formula (I) for use in the prophylaxis and/or treatment of sarcoïdosis and/or a sarcoïdosis-related condition.

The present invention also provides pharmaceutical compositions comprising a compound of the invention, and a suitable pharmaceutical carrier, excipient or diluent for use in the prophylaxis and/or treatment of sarcoïdosis and/or a sarcoïdosis-related condition.

In a further particular aspect, the pharmaceutical composition may additionally comprise further therapeutically active ingredients suitable for use in combination with the compounds of the invention. In a more particular aspect, the further therapeutically active ingredient is an agent for the treatment of sarcoïdosis and/or a sarcoïdosis-related condition.

In one aspect of the invention, this invention provides a method for the prophylaxis and/or treatment of sarcoïdosis and/or a sarcoïdosis-related condition in a mammal in need thereof, e.g. in particular humans, which method comprises administering an effective amount of a pharmaceutical composition or compound of the invention as described herein.

In one aspect, this invention provides a compound of the invention for use in the prophylaxis and/or treatment of sarcoïdosis and/or a sarcoïdosis-related condition in a mammal, in particular humans, afflicted with sarcoïdosis, wherein the administration of said compound results in a detectable improvement in one or more symptoms of said sarcoïdosis. In one aspect said symptom is one or more of formation of granulomas, fatigue, weight loss, fever, aches, pains, arthritis, dry eyes, swelling of the knees, blurry vision, shortness of breath, cough and skin lesions

The present invention also provides pharmaceutical compositions comprising the compound of the invention, and a suitable pharmaceutical carrier, excipient or diluent for use in medicine. In a particular aspect, the pharmaceutical composition is for use in the prophylaxis and/or treatment of sarcoïdosis and/or a sarcoïdosis-related condition.

In additional aspects, this invention provides methods for synthesizing the compound of the invention, with representative synthetic protocols and pathways disclosed later on herein.

The present invention also provides methods for the production of these pharmaceutical compositions of the invention and methods for the prophylaxis and/or treatment of sarcoïdosis and/or a sarcoïdosis-related condition by administering the pharmaceutical compositions of the invention.

Accordingly, in a first aspect of the invention, a compound of the invention is provided having a Formula (I) for use in the prophylaxis and/or treatment of sarcoïdosis and/or a sarcoïdosis-related condition.

The present invention also provides pharmaceutical compositions comprising a compound of the invention, and a suitable pharmaceutical carrier, excipient or diluent for use in the prophylaxis and/or treatment of sarcoïdosis and/or a sarcoïdosis-related condition.

In a further particular aspect, the pharmaceutical composition may additionally comprise further therapeutically active ingredients suitable for use in combination with the compounds of the invention. In a more particular aspect, the further therapeutically active ingredient is an agent for the treatment of sarcoïdosis and/or a sarcoïdosis-related condition.

In one aspect of the invention, this invention provides a method for the prophylaxis and/or treatment of sarcoïdosis and/or a sarcoïdosis-related condition in a mammal in need thereof, e.g. in particular humans, which method comprises administering an effective amount of a pharmaceutical composition or compound of the invention as described herein.

In one aspect, this invention provides a compound of the invention for use in the prophylaxis and/or treatment of sarcoïdosis and/or a sarcoïdosis-related condition in a mammal, in particular humans, afflicted with sarcoïdosis, wherein the administration of said compound results in a detectable improvement in one or more symptoms of said sarcoïdosis. In one aspect said symptom is one or more of formation of granulomas, fatigue, weight loss, fever, aches, pains, arthritis, dry eyes, swelling of the knees, blurry vision, shortness of breath, cough and skin lesions

The present invention also provides pharmaceutical compositions comprising the compound of the invention, and a suitable pharmaceutical carrier, excipient or diluent for use in medicine. In a particular aspect, the pharmaceutical composition is for use in the prophylaxis and/or treatment of sarcoïdosis and/or a sarcoïdosis-related condition.

In additional aspects, this invention provides methods for synthesizing the compound of the invention, with representative synthetic protocols and pathways disclosed later on herein.

The present invention provides the compounds of the invention, e.g. a compound according to Formula I (Compound 1), for use in the prophylaxis and/or treatment of giant cell arteritis. In particular, the compounds of the invention may act as inhibitors of JAK, and more particularly of JAK1.

Furthermore, the present invention provides pharmaceutical compositions comprising a compound of the invention, e.g. a compound according to Formula I (Compound 1), for use in the prophylaxis and/or treatment of giant cell arteritis.

The present invention also provides methods for the production of these pharmaceutical compositions of the invention and methods for the prophylaxis and/or treatment of giant cell arteritis by administering the pharmaceutical compositions of the invention.

Accordingly, in another aspect of the invention, a compound of the invention is provided according to Formula (I) for use in the prophylaxis and/or treatment of giant cell arteritis.

The present invention also provides pharmaceutical compositions comprising a compound of the invention, and a suitable pharmaceutical carrier, excipient or diluent for use in the prophylaxis and/or treatment of giant cell arteritis.

In a further particular aspect, a pharmaceutical composition comprising a compound of the invention may additionally comprise further therapeutically active ingredients suitable for use in combination with the compounds of the invention. In a more particular aspect, the further therapeutically active ingredient is an agent for the treatment of giant cell arteritis.

In one aspect of the invention, this invention provides a method for the prophylaxis and/or treatment of giant cell arteritis in a mammal in need thereof, in particular humans, which method comprises administering an effective amount of a pharmaceutical composition or compound of the invention as described herein.

In one aspect, this invention provides a compound of the invention for use in the prophylaxis and/or treatment of giant cell arteritis in a mammal, in particular humans, afflicted with giant cell arteritis.

The present invention also provides pharmaceutical compositions comprising a compound of the invention, and a suitable pharmaceutical carrier, excipient or diluent for use in medicine. In a particular aspect, the pharmaceutical composition is for use in the prophylaxis and/or treatment of giant cell arteritis.

Other objects and advantages will become apparent to those skilled in the art from a consideration of the ensuing detailed description.

Moreover, the compounds of the invention, useful in the pharmaceutical compositions and treatment methods disclosed herein, are pharmaceutically acceptable as prepared and used.

It will be appreciated that the compound of the invention may be metabolized to yield biologically active metabolites.

### DETAILED DESCRIPTION OF THE INVENTION

### Definitions

The following terms are intended to have the meanings presented therewith below and are useful in understanding the description and intended scope of the present invention.

When describing the invention, which may include compounds, pharmaceutical compositions comprising such compounds and methods of using such compounds and compositions, the following terms, if present, have the following meanings unless otherwise indicated.

The articles ‘a’ and ‘an’ may be used herein to refer to one or to more than one (i.e. at least one) of the grammatical objects of the article. By way of example ‘an analogue’ means one analogue or more than one analogue.

‘Pharmaceutically acceptable’ means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

‘Pharmaceutically acceptable salt’ refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzene sulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term ‘pharmaceutically acceptable cation’ refers to an acceptable cationic counter-ion of an acidic functional group. Such cations are exemplified by sodium, potassium, calcium, magnesium, ammonium, tetraalkylammonium cations, and the like.

‘Pharmaceutically acceptable vehicle’ refers to a diluent, adjuvant, excipient or carrier with which a compound of the invention is administered.

‘Solvate’ refers to forms of the compound that are associated with a solvent, usually by a solvolysis reaction. This physical association includes hydrogen bonding. Conventional solvents include water, EtOH, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

‘Subject’ includes humans and non-human animals. The terms ‘patient’ and ‘subject’ are used interchangeably herein.

‘Effective amount’ means the amount of a compound of the invention that, when administered to a subject for treating a disease, is sufficient to effect such treatment for the disease. The “effective amount” can vary depending on the compound, the disease and its severity, and the age, weight, etc., of the subject to be treated.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; the administration of an anti-malarial agent such as chloroquine, in advance of a visit to a geographical region where malaria is endemic or the risk of contracting malaria is high.

‘Treating’ or ‘treatment’ of any disease or disorder refers, in one embodiment, to ameliorating the disease or disorder (i.e. arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g. stabilization of a discernible symptom), physiologically, (e.g. stabilization of a physical parameter), or both. In a further embodiment, “treating” or “treatment” relates to slowing the progression of the disease.

As used herein, the term ‘chronic’ in a chronic condition, refers to a condition or disease that is persistent, and/or long-lasting in the effects it produces. In particular, the term refers to a condition or disease that persists over a period of greater than 4 weeks, or at least 8 weeks, or at least 12 weeks, or at least 16 weeks, or at least 20 weeks, or at least 24 weeks.

As used herein, the term ‘CRP’ refers to the C-Reactive protein in blood serum and is a marker of inflammation. In particular guidelines for CRP are widely available, and, and normal values of <0.5 mg/dL are recommended (The Merck Manual of Diagnosis and Therapy, 2011).

As used herein, the term ‘ESR’ refers to the erythrocyte sedimentation rate in blood and is a marker of inflammation. In particular guidelines for ESR are widely available, and normal values of 0-20 mm/h in female and 0-15 mm/h are recommended (The Merck Manual of Diagnosis and Therapy, 2011).

‘Compound(s) of the invention’, and equivalent expressions, are meant to embrace compounds of the Formula(e) as herein described, which expression includes the pharmaceutically acceptable salts, and the solvates, e.g. hydrates, and the solvates of the pharmaceutically acceptable salts where the context so permits. Similarly, reference to intermediates, whether or not they themselves are claimed, is meant to embrace their salts, and solvates, where the context so permits.

As used herein, the term ‘JAK’ refers to Janus kinase, a family of tyrosine kinases that transduce cytokine-mediated signals via the JAK-STAT pathway. In particular, the JAK family comprises four members Janus Kinase 1 (JAK1), Janus Kinase 2 (JAK2), Janus Kinase 3 (JAK3), and Tyrosine Kinase 2 (TYK2).

As used herein the term ‘SALT score’ or ‘Severity Alopecia Tool’ refers to a clinical scoring used to determine the severity of the disease, as described in (Olsen et al., 2004). The score is divided in 6 levels, namely S₀ (no hair loss), S₁ (<25% hair loss), S₂ (25-49% hair loss), S₃ (50-74% hair loss), S₄ (75-99% hair loss), and S₅ (100% hair loss). To determine the SALT, the scalp of an individual is decomposed in 4 areas: left side (18% of the overall head surface), the right side (18% of the overall head surface), the top (40% of the overall head surface) and the back (24% of the overall head surface). Change in SALT score from baseline incorporates density as well as extent into scoring system (for example: SALT 75−SALT 50=SALT 25. The percent scalp hair regrowth between 2 time points t1 and t2 based on SALT score is determined as follows (SALTt2−SALTt1)/SALTt2=tlt2%. The regrowth or SALTₜ₁ₜ₂ where the subscript equals the percent change in SALT score. For example (SALT75-SALT50)/SALT75=33%. The resulting regrowth improvement is SALT₃₃.

A global assessment A is then made on the overall improvement, taking into account the extent and density of regrowth by the SALT scoring system, wherein A₀=no change or further loss, A₁=1-24% regrowth, A₂=25-49% regrowth, A₃=50-74% regrowth, A₄=75-99% regrowth, and A₅=100% regrowth

As used herein, the term ‘isotopic variant’ refers to a compound that contains unnatural proportions of isotopes at one or more of the atoms that constitute such compound. For example, an ‘isotopic variant’ of a compound can contain one or more non-radioactive isotopes, such as for example, deuterium (²H or D), carbon-13 (¹³C), nitro (′⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

As used herein the term ‘VAST score’ or ‘Vitiligo Area Scoring Index’ refers to a clinical scoring used to determine the severity of the disease, as described in (Hamzavi I et al., 2004). This score is used to evaluate vitiligo parametrically, and the lower the VASI score, the lower the severity of vitiligo. The body is divided into 5 separate and mutually exclusive regions: hands, upper extremities (excluding hands), trunk, lower extremities (excluding the feet), and feet. The axilliary and inguinal regions are included with the upper and lower extremities, respectively, while the buttocks are included with the lower extremities. The face and neck areas are assessed but not included in the overall evaluation. One hand unit, which encompasses the palm plus the volar surface of all the digits, is approximately 1% of the total body surface area and is used as a guide to estimate the baseline percentage of vitiligo involvement of each body region. At each follow-up assessment, any macular repigmentation is noted, and the extent of residual depigmentation within each affected patch that had been present at baseline is estimated to the nearest of 1 of the following percentages: 0, 10%, 25%, 50%, 75%, 90%, or 100%. Any new depigmented patches that developed during the study are also estimated using the hand unit method and are included in the VASI calculation. For each body region, the VASI is determined by the product of the area of vitiligo in hand units (which was set at 1% per unit) and the extent of depigmentation within each hand unit-measured patch (possible values of 0, 10%, 25%, 50%, 75%, 90%, or 100%). The total body VASI was then calculated using the following formula by considering the contributions of all body regions (possible range, 0-100):

$$VASI = {\sum }_{all body site} [ Hand Unit ] \times [ Residual Depigmentation ]$$

As used herein, the term ‘Cutaneous Lupus Erythematosus Disease Area and Severity Index’ or ‘CLASI’ refers to a clinical scoring used to determine the severity of the disease, as described in (Albrecht et al, 2005). This index takes into account lesional morphology as well as anatomic location, and is used by both dermatologists and rheumatologists. The lower the CLASI score, the lower the severity of the disease. In particular, mild CL is determined by a CLASI score of 0-9, moderate CL is determined by a CLASI score of 10-20, and severe CL is determined by a CLASI score of 21-70.

As used herein, the terms “cutaneous lupus” (CL) or “cutaneous lupus erythematosus” (CLE) are used interchangeably and refer to a group of diseases affecting the skin and subcutaneous tissue, classified under the WHO International Classification of Diseases (ICD-10) as code L93. As used herein the term “cutaneous lupus” includes acute cutaneous lupus erythematosus (ACLE), subacute cutaneous lupus erythematosus (SCLE), and chronic cutaneous lupus erythematosus (CCLE) and excludes systemic lupus erythematosus (SLE).

As used herein the terms “acute cutaneous lupus erythematosus” or “acute cutaneous lupus” or “ACLE” are used interchangeably to refer to a form of cutaneous lupus that is often characterized as a photosensitive dermatosis. It can appear as flattened areas of red skin that resemble a persistent sunburn or have a rash-like appearance. ACLE may erupt in a butterfly pattern localized to the central portion of the face and/or in a generalized pattern including other areas such as the arms, legs and body.

The term “subacute cutaneous lupus erythematosus” or “subacute cutaneous lupus” or “SCLE” are used interchangeably to refer to a non-scarring non-atrophy-producing photosensitive dermatosis. In some cases, SCLE refers to a condition characterized by a non-itchy ring-shaped dry rash on the upper back and chest, often following sun exposure. SCLE is often characterized by two forms including papulosquamous lesions and annular lesions. Papulosquamous lesions often appear as red scaly patches that look psoriasiform. Annular lesions are ring-shaped with a small amount of scale on the edge of the lesions. These lesions do not itch and often appear on the chest as well as the upper back and neck, but also may also be seen on the face and arms.

The terms “chronic cutaneous lupus erythematosus” or “chronic cutaneous lupus” or “CCLE” or “discoid lupus erythematosus (DLE)” are used interchangeably to refer to a chronic, scarring, atrophy producing, photosensitive dermatosis. It commonly appears as red scaly patches which leave white scars. DLE predominantly affects the cheeks and nose, but sometimes involves the upper back, neck, backs of hands, bald areas in scalp and the lips. Verrucous DLE, lupus profundus, mucosal DLE, palmar-plantar DLE and lupus tumidus are some specific forms of DLE. Verrucous DLE refers to DLE having lesions that can develop into very thick scales. Lupus profundus refers to DLE having lesions that may occur in conjunction with firm lumps in the fatty tissue underlying the skin. Mucosal DLE refers to the lesions that occasionally occur in the mucus membranes of the mouth, nose and eyes. Palmar-plantar DLE refers to the lesions that occasionally occur on the hands and feet. Lupus tumidus refers to DLE having smooth, shiny, red-violet plaques of the head and neck that can be pruritic and have a fine scale.

As used here in the term “lupus nephritis” (LN) refers to an inflammation of the kidneys caused by systemic lupus erythematosus (SLE). Lupus nephritis includes, but not limited to minimal mesangial glomerulonephritis, mesangial proliferative glomerulonephritis, focal glomerulonephritis, diffuse proliferative nephritis, membranous lupus nephritis, and advanced sclerosing lupus nephritis.

The term “minimal mesangial glomerulonephritis” (Class I disease) refers to a subtype of lupus nephritis characterized histologically by a normal appearance under a light microscope, but having visible mesangial deposits under an electron microscope.

The term “mesangial proliferative glomerulonephritis” (Class II disease) refers to a subtype of lupus nephritis characterized by mesangial hypercellularity and matrix expansion and possible visible microscopic haematuria with or without proteinuria.

The term “focal glomerulonephritis” (Class III disease) refers to a subtype of lupus nephritis characterized by sclerotic lesions involving less than 50% of the glomeruli, which can be segmental or global, and active or chronic, with endocapillary or extracapillary proliferative lesions.

The term “diffuse proliferative nephritis” (Class IV disease) refers to a subtype of lupus nephritis characterized by involvement of more than 50% of glomeruli. Lesions can be segmental or global, and active or chronic, with endocapillary or extracapillary proliferative lesions. Under electron microscopy, subendothelial deposits can be noted, and some mesangial changes may be present.

The term “membranous glomerulonephritis” or “membranous lupus nephritis” (Class V disease) refers to a subtype of lupus nephritis characterized by diffuse thickening of the glomerular capillary wall (segmentally or globally), with diffuse membrane thickening, and subepithelial deposits seen under the electron microscope. Clinically, membranous lupus nephritis presents with signs of nephrotic syndrome. Microscopic haematuria and hypertension may also been seen. Membranous lupus nephritis also can lead to thrombotic complications such as renal vein thromboses or pulmonary emboli.

The term “advanced sclerosing lupus nephritis” (Class VI disease) refers to a subtype of lupus nephritis characterized by global sclerosis involving more than 90% of glomeruli, and representing healing of prior inflammatory injury.

As used herein the term “sarcoïdosis” refers to a group of conditions comprising systemic sarcoïdosis, cutaneous sarcoïdosis, Löfgren's syndrome, neurosarcoïdosis, pulmonary sarcoïdosis, cardiac sarcoïdosis, ocular sarcoïdosis, hepatic sarcoïdosis, musculoskeletal sarcoïdosis, renal sarcoïdosis, or sarcoïdosis with the involvement of other organs or tissues.

The term “sarcoïdosis-related condition” refers to diseases, conditions and disorders that are otherwise related and/or caused by sarcoïdosis.

The term “systemic sarcoïdosis” refers to sarcoïdosis with multiple organ involvement. In some embodiments, symptoms of systemic sarcoïdosis comprise non-specific general symptoms such as weight loss, fatigue, loss of appetite, fever, chills, night sweats, formation of granulomas, fatigue, aches, pains or arthritis.

The term “cutaneous sarcoïdosis” refers to a complication of sarcoïdosis with skin involvement. More specifically, the cutaneous sarcoïdosis comprises annular sarcoïdosis, erythrodermic sarcoïdosis, ichthyosiform sarcoïdosis, hypopigmented sarcoïdosis, morpheaform sarcoïdosis, mucosal sarcoïdosis, papular sarcoid, scar sarcoid, subcutaneous sarcoïdosis and ulcerative sarcoïdosis. In some embodiments, the one or more symptoms of cutaneous sarcoïdosis comprise a variety of skin lesions or conditions, either specific or non-specific. Exemplary skin lesions or conditions associated with cutaneous sarcoïdosis comprise papules, skin plaques, lupus pernio, raised red firm skin sores, cellulitis, furunculosis, other inflammatory panniculitis, maculopapular eruptions, nodular lesions deeper in the skin, skin rashes, skin lesions and hair loss.

The term “Löfgren's syndrome” refers to an acute presentation of systemic sarcoïdosis, which is commonly characterized by the triad of erythema nodosum, bilateral hilar denopathy and arthritis or arthralgias. It may also be accompanied by fever.

The term “neurosarcoïdosis” refers to sarcoïdosis with the involvement of any part of the nervous system.

The term “pulmonary sarcoïdosis” refers to sarcoïdosis that affects pulmonary tissues or organs, more specifically lungs. The symptoms of pulmonary sarcoïdosis usually involve lung and/or chest symptoms. In some embodiments, pulmonary sarcoïdosis can develop into pulmonary fibrosis, which can distort the structure of the lungs and impair breathing or bronchiectasis. Pulmonary fibrosis is a lung disease characterized by destruction and widening of the large airways. In a specific embodiment, said disease or disorder is pulmonary fibrosis.

The term “cardiac sarcoïdosis” refers to sarcoïdosis with involvement of the myocardium.

THE INVENTION

The present invention relates to a compound useful in the prophylaxis and/or treatment of alopecia areata. In particular, a compound of the invention inhibits JAK. More particularly, a compound of the invention inhibits JAK1. The present invention also provides methods for the prophylaxis and/or treatment of alopecia areata comprising administering a compound of the invention to a subject in need thereof.

The present invention also provides pharmaceutical compositions comprising said compounds and methods for the prophylaxis and/or treatment of alopecia areata by administering the compounds of the invention.

Accordingly, in a first aspect of the invention, is provided a compound of the invention for use is the prophylaxis and/or treatment of alopecia areata, wherein said compound of the invention is according to Formula (I)

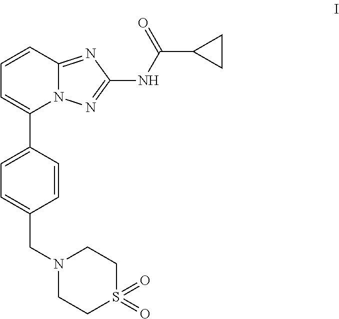 [SMILES: O=C(Nc1nc2cccc(-c3ccc(CN4CCS(=O)(=O)CC4)cc3)n2n1)C1CC1]

In one embodiment, is provided a metabolite of the compound of Formula (I) for use is the prophylaxis and/or treatment of alopecia areata, said metabolite being a compound according to Formula (II):

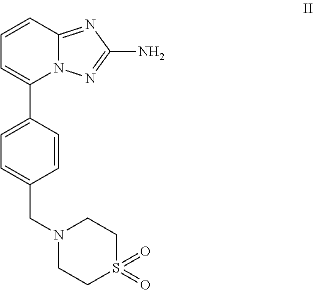 [SMILES: Nc1nc2cccc(-c3ccc(CN4CCS(=O)(=O)CC4)cc3)n2n1]

The present invention further relates to a compound useful in the prophylaxis and/or treatment of vitiligo. In particular, a compound of the invention inhibits JAK. More particularly, a compound inhibits JAK1. The present invention also provides methods for the prophylaxis and/or treatment of vitiligo comprising administering a compound of the invention to a subject in need thereof.

The present invention also provides pharmaceutical compositions comprising said compounds and methods for the prophylaxis and/or treatment of vitiligo by administering the compounds of the invention.

Accordingly, in a first aspect of the invention, is provided a compound of the invention for use is the prophylaxis and/or treatment of vitiligo, wherein said compound of the invention is according to Formula (I)

In another embodiment, the compound of the invention is a metabolite of the compound according to Formula I, said metabolite being a compound according to Formula II:

The present invention also relates to a compound useful in the prophylaxis and/or treatment of lupus nephritis. In particular, a compound of the invention inhibits JAK. More particularly, a compound of the invention inhibits JAK1. The present invention also provides methods for the prophylaxis and/or treatment of lupus nephritis comprising administering a compound of the invention to a subject in need thereof.

The present invention further provides pharmaceutical compositions comprising said compounds and methods for the prophylaxis and/or treatment of lupus nephritis by administering the compounds of the invention.

Accordingly, in a first aspect of the invention, a compound of the invention is provided for use is the prophylaxis and/or treatment of lupus nephritis, wherein said compound of the invention is according to Formula (I).

In one embodiment, is provided a metabolite of the compound of Formula (I) for use is the prophylaxis and/or treatment of lupus nephritis, said metabolite being according to Formula (II):

The present invention also relates to a compound useful in the prophylaxis and/or treatment of sarcoïdosis and/or a sarcoïdosis-related condition. In particular, a compound of the invention inhibits JAK. More particularly, a compound of the invention inhibits JAK1. The present invention also provides methods for the prophylaxis and/or treatment of sarcoïdosis and/or a sarcoïdosis-related condition comprising administering a compound of the invention to a subject in need thereof

The present invention also provides pharmaceutical compositions comprising said compound and methods for the prophylaxis and/or treatment of sarcoïdosis and/or a sarcoïdosis-related condition by administering the compounds of the invention.

Accordingly, in a first aspect of the invention, is provided a compound of the invention for use is the prophylaxis and/or treatment of sarcoïdosis and/or a sarcoïdosis-related condition, wherein said compound of the invention is according to Formula (I).

In one embodiment, is provided a metabolite of the compound of Formula (I) for use is the prophylaxis and/or treatment of sarcoïdosis and/or a sarcoïdosis-related condition, said metabolite being according to Formula (II).

The present invention also relates to a compound useful in the prophylaxis and/or treatment of giant cell arteritis. In particular, a compound of the invention inhibits JAK. More particularly, a compound of the invention inhibits JAK1. The present invention also provides methods for the prophylaxis and/or treatment of giant cell arteritis comprising administering a compound of the invention to a subject in need thereof.

The present invention also provides pharmaceutical compositions comprising said compounds and methods for the prophylaxis and/or treatment of giant cell arteritis by administering the compounds of the invention.

Accordingly, in a first aspect of the invention, is provided a compound of the invention for use is the prophylaxis and/or treatment of giant cell arteritis, wherein said compound of the invention is according to Formula (I).

In one embodiment, a compound of the invention is a metabolite of the compound according to Formula (I), said metabolite being according to Formula II.

In one embodiment a compound of the invention is not an isotopic variant.

In one aspect a compound of the invention according to any one of the embodiments herein described is present as the free base.

In one aspect a compound of the invention according to any one of the embodiments herein described is a pharmaceutically acceptable salt. In a particular embodiment, a compound of the invention is a salt wherein said salt is formed with a salt forming agent selected from a hydrobromic acid, hydrochloric acid, sulfuric acid, toluenesulfonic acid, benzenesulfonic acid, oxalic acid, maleic acid, naphthalene-2-sulfonic acid, naphthalene-1,5-disulfonic acid, 1-2-ethane disulfonic acid, methanesulfonic acid, 2-hydroxy ethanesulfonic acid, phosphoric acid, ethane sulfonic acid, malonic acid, 2-5-dihydroxybenzoic acid, or L-Tartaric acid. In a more particular embodiment, a compound of the invention is a maleic acid salt.

In one aspect a compound of the invention according to any one of the embodiments herein described is a solvate of the compound.

In one aspect a compound of the invention according to any one of the embodiments herein described is a solvate of a pharmaceutically acceptable salt of a compound. In a particular embodiment, the salt of a solvate is a [Compound according to Formula I:HCl:3H₂O] adduct.

In one embodiment, the present invention provides a compound of the invention or pharmaceutical compositions comprising a compound of the invention, for use in the prophylaxis and/or treatment of alopecia areata when dosed orally at a dose of between 25 mg to 400 mg, administered once or twice a day. In particular, the compound of the invention is dosed orally at a dose of between 100 mg to 250 mg. In particular, the dose is selected from 25 mg twice per day (b.i.d.), 50 mg once a day (q.d.), 50 mg b.i.d., 100 mg q.d., 100 mg b.i.d., and 200 mg q.d.

In another embodiment, the present invention provides a compound of the invention or pharmaceutical composition comprising a compound of the invention, for use in the prophylaxis and/or treatment of alopecia areata, wherein the administration of said compound, or a pharmaceutically acceptable salt, solvate, or polymorph thereof, or a pharmaceutical composition results in detectable changes of at least one alopecia-associated biomarker.

In particular, said alopecia-associated biomarker level is decreased by at least 5%, at least 10%, at least 15%, at least 20%, at least 30%, at least 40%, at least 50%, at least 60%, at least 70%, at least 80%, at least 90% compared to pre-treatment levels. More particularly, said alopecia-associated biomarker level is decreased by at least 5%, at least 10%, or at least 15% compared to pre-treatment levels. In a more particular embodiment, the alopecia-associated biomarker is IFN_{γ}.

In a further more particular embodiment, the alopecia areat-associated biomarker is IFN_{γ} wherein the IFN_{γ} levels are decreased by at least 5% after 12 weeks compared to pre-treatment levels.

In another embodiment, the present invention provides the compound of the invention or pharmaceutical compositions comprising a compound of the invention, for use in the prophylaxis and/or treatment of vitiligo, wherein the administration of said compound, or a pharmaceutically acceptable salt thereof, or a pharmaceutical composition results in detectable changes of at least one vitiligo-associated biomarker.

In particular, said vitiligo-associated biomarker level is decreased by at least 5%, at least 10%, at least 15%, at least 20%, at least 30%, at least 40%, at least 50%, at least 60%, at least 70%, at least 80%, at least 90% compared to pre-treatment levels. More particularly, said vitiligo-associated biomarker level is decreased by at least 5%, at least 10%, or at least 15% compared to pre-treatment levels. In a more particular embodiment, the vitiligo-associated biomarker is IFN_{γ}.

In a further more particular embodiment, the vitiligo-associated biomarker is IFN_{γ} wherein the IFN_{γ} levels are decreased by at least 5% after 12 weeks compared to pre-treatment levels.

In another embodiment, the present invention provides a compound of the invention or pharmaceutical compositions comprising a compound of the invention, for use in the prophylaxis and/or treatment of lupus nephritis, wherein the administration of said compound, or a pharmaceutically acceptable salt, solvate, or polymorph thereof, or a pharmaceutical composition results in detectable changes of at least one lupus nephritis-associated biomarker. In particular, said lupus nephritis-associated biomarker levels is decreased by at least 5%, at least 10%, at least 15%, at least 20%, at least 30%, at least 40%, at least 50%, at least 60%, at least 70%, at least 80%, at least 90% compared to pre-treatment levels. More particularly, said lupus nephritis-associated biomarker levels is decreased by at least 5%, at least 10%, or at least 15% compared to pre-treatment levels.

In a particular embodiment, the lupus nephritis-associated biomarker is selected from IL-6, IL-10, IFNγ and TGFβ. In a more particular embodiment, the lupus nephritis-associated biomarker is IL-6.

In another embodiment, the present invention provides a compound of the invention or pharmaceutical compositions comprising a compound of the invention, for use in the prophylaxis and/or treatment of sarcoïdosis and/or a sarcoïdosis-related condition, wherein the administration of said compound, or a pharmaceutically acceptable salt, solvate, or polymorph thereof, or a pharmaceutical composition results in detectable changes of at least one sarcoïdosis and/or a sarcoïdosis-related condition associated biomarker. In particular, said sarcoïdosis and/or a sarcoïdosis-related condition associated biomarker levels is decreased by at least 5%, at least 10%, at least 15%, at least 20%, at least 30%, at least 40%, at least 50%, at least 60%, at least 70%, at least 80%, at least 90% compared to pre-treatment levels. More particularly, said sarcoïdosis and/or a sarcoïdosis-related condition associated biomarker levels is decreased by at least 5%, at least 10%, or at least 15% compared to pre-treatment level.

In a particular embodiment, the sarcoïdosis and/or a sarcoïdosis-related condition associated biomarker is CXCL9 or CXCL10. In a more particular embodiment, the sarcoïdosis and/or a sarcoïdosis-related condition associated biomarker is CXCL10.

In another embodiment, the present invention provides a compound of the invention or pharmaceutical compositions comprising a compound of the invention, for use in the prophylaxis and/or treatment of giant cell arteritis, wherein the administration of said compound, or a pharmaceutically acceptable salt thereof, or a pharmaceutical composition results in detectable changes of at least one giant cell arteritis-associated biomarker. In particular, said giant cell arteritis-associated biomarker levels is decreased by at least 5%, at least 10%, at least 15%, at least 20%, at least 30%, at least 40%, at least 50%, at least 60%, at least 70%, at least 80%, at least 90% compared to pre-treatment levels. More particularly, said giant cell arteritis-associated biomarker levels is decreased by at least 5%, at least 10%, or at least 15% compared to pre-treatment levels. In a particular embodiment, the giant cell arteritis-associated biomarker is IL1, IL6 and/or GM-CSF

### CLAUSES

1. A compound according to Formula I:

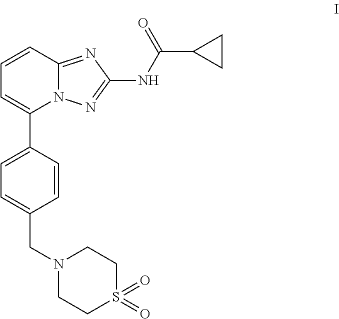 [SMILES: O=C(Nc1nc2cccc(-c3ccc(CN4CCS(=O)(=O)CC4)cc3)n2n1)C1CC1]

or a pharmaceutically acceptable salt thereof, or a solvate or the salt of a solvate thereof, or an active metabolite thereof for use in the prophylaxis and/or treatment of alopecia areata, vitiligo, cutaneous lupus, lupus nephritis, giant cell arteritis, sarcoïdosis and/or a sarcoïdosis-related condition.
2. The compound for use according to clause 1, wherein the active metabolite is according to Formula II:

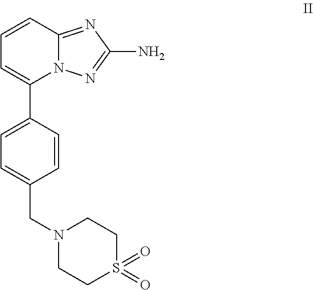 [SMILES: Nc1nc2cccc(-c3ccc(CN4CCS(=O)(=O)CC4)cc3)n2n1]

3. A compound for use according to clause 1, wherein the compound according to Formula I is a pharmaceutically acceptable salt, wherein said salt is formed with a salt forming agent selected from a hydrobromic acid, hydrochloric acid, sulfuric acid, toluenesulfonic acid, benzenesulfonic acid, oxalic acid, maleic acid, naphthalene-2-sulfonic acid, naphthalene-1,5-disulfonic acid, 1-2-ethane disulfonic acid, methanesulfonic acid, 2-hydroxy ethanesulfonic acid, phosphoric acid, ethane sulfonic acid, malonic acid, 2-5-dihydroxybenzoic acid, or L-Tartaric acid.
4. A compound for use according to clause 1, wherein the compound according to Formula I is a pharmaceutically acceptable salt, wherein said salt is formed with maleic acid.
5. A compound, or a pharmaceutically acceptable salt thereof for use according to any one of clauses 1-4, in combination with a further therapeutic agent.
6. A pharmaceutical composition for use in the prophylaxis and/or treatment of alopecia areata, comprising the compound according to Formula I, or a pharmaceutically acceptable salt thereof, and a pharmaceutically acceptable carrier, excipient, or diluent.
7. A pharmaceutical composition for use according to clause 6, comprising a further therapeutic agent.
8. The compound or a pharmaceutically acceptable salt thereof, according to clause 5 or the pharmaceutical composition for use according to clause 7, wherein the further therapeutic agent is an agent for the prophylaxis and/or treatment of alopecia areata.
9. The compound or a pharmaceutically acceptable salt thereof, according to clause 5 or the pharmaceutical composition for use according to clause 7, wherein the further therapeutic agent is selected from clobetasol propionate, desoximetasone, hydrocortisone, methylprednisolone, prednisone, prednisolone, budesonide, dexamethasone, dinitrochlorobenzene, squaric acid dibutylester, diphenylcyclopropenone, topical minoxidil, anthralin, psoralen cyclosporin, azathioprine, and methotrexate.
10. A compound, or a pharmaceutically acceptable salt thereof, for use according to any one of clauses 1-5, or a pharmaceutical composition according to clause 6 or 7 in an individual afflicted with alopecia areata.
11. A compound, or a pharmaceutically acceptable salt thereof, or a pharmaceutical composition for use according to any one of clauses 1-10, in an individual afflicted with alopecia areata, as characterized by the Severity Alopecia Tool (SALT) score.
12. A compound, or a pharmaceutically acceptable salt thereof, or a pharmaceutical composition for use according to any one of clauses 1-10 in an individual afflicted with alopecia areata, as characterized by a SALT score of at least 25%.
13. A compound, or a pharmaceutically acceptable salt thereof, or a pharmaceutical composition for use according to any one of clauses 1-10 in an individual afflicted with alopecia areata, as characterized by a SALT score of at least 50%.
14. A compound, or a pharmaceutically acceptable salt thereof, or a pharmaceutical composition for use according to any one of clauses 1-10 in an individual afflicted with alopecia areata, as characterized by a SALT score of at least 75%.
15. A compound, or a pharmaceutically acceptable salt thereof, or a pharmaceutical composition for use according to any one of clauses 1-10, wherein the regrowth improvement is at least 10% (SALT₁₀), 20% (SALT₂₀), 30% (SALT₃₀), 40% (SALT₄₀), or 50% (SALT₅₀) after treatment compared to before treatment.
16. A method for the prophylaxis and/or treatment of alopecia areata comprising the steps of:
   measuring the SALT score of an individual by performing a scalp hair loss analysis by assessing the hair loss on the left side, right side, top and back of the patient, wherein the left side and the right side each account for 18% of the overall head surface, the top accounts for 40% of the overall head surface and the back accounts for 24% of the overall head surface,
      assessing the type of hair remaining on each area (pigmented/non-pigmented/indeterminant),
   calculating a SALT score by the formula:

SALT score=(% left side)*0.18+(% right side)*0.18+(% top side)*0.40+(% back side)*0.24, and
determining a daily dose of the compound according to Formula I, or a pharmaceutically acceptable salt thereof comprised between 25 mg and 400 mg for administration to said individual.
17. A compound, or a pharmaceutically acceptable salt thereof, or a pharmaceutical composition for use according to any one of clauses 1-10, wherein the administration of said compound, or a pharmaceutically acceptable salt thereof, or a pharmaceutical composition results in detectable changes of at least one alopecia-associated biomarker.
18. A compound, or a pharmaceutically acceptable salt thereof, or a pharmaceutical composition for use according to any one of clauses 1-10 in an individual afflicted with alopecia areata, said use comprising the steps of:
   a) assaying a blood sample,
   b) measuring levels of at least one alopecia areata-associated biomarker,
   c) administering a therapeutically effective amount of the compound, or a pharmaceutically acceptable salt thereof to the individual,
   d) measuring the post-treatment level of said alopecia areata-associated biomarker and comparing with pre-treatment levels, and
   e) adjusting the dosage of the compound, or a pharmaceutically acceptable salt thereof to be administered to the individual, wherein said dosage is increased when a decrease of less than 2% after 12 weeks in levels of said alopecia areata-associated biomarker is detected.
19. A compound, or a pharmaceutically acceptable salt thereof, for use according to clause 18, wherein the biomarker is IFN_{γ}.
20. A compound, or a pharmaceutically acceptable salt thereof, for use according to clause 19, wherein the biomarker level is decreased by at least 5%, at least 10%, or at least 15% compared to pre-treatment levels.
21. A compound, or a pharmaceutically acceptable salt thereof, or a pharmaceutical composition for use according to clause 19, wherein the IFN_{γ} are decreased by at least 5% compared to pre-treatment levels after 12 weeks of treatment.
22. A pharmaceutical composition for use in the prophylaxis and/or treatment of vitiligo, comprising the compound according to Formula I, or a pharmaceutically acceptable salt thereof, and a pharmaceutically acceptable carrier, excipient, or diluent.
23. A pharmaceutical composition for use according to clause 22, comprising a further therapeutic agent.
24. The compound or a pharmaceutically acceptable salt thereof, according to clause 5 or the pharmaceutical composition for use according to clause 23, wherein the further therapeutic agent is an agent for the prophylaxis and/or treatment of vitiligo.
25. The compound or a pharmaceutically acceptable salt thereof, according to clause 5 or the pharmaceutical composition for use according to clause 23, wherein the further therapeutic agent is selected from topical, corticosteroids, methotrexate, cyclosporine, retinoids, tioguanine, hydroxyurea, sulfasalazine, mycophenolate mofetil, azathioprine, tacrolimus, fumaric acid esters or biologics.
26. A compound, or a pharmaceutically acceptable salt thereof, for use according to any one of clauses 1-5, or a pharmaceutical composition according to clause 22 or 23 in an individual afflicted with vitiligo.
27. A compound, or a pharmaceutically acceptable salt thereof, or a pharmaceutical composition for use according to any one of clauses 1-5 and 22-26 in an individual afflicted with vitiligo, as characterized by the Vitiligo Activity Severity Index (VAST) score.
28. A compound, or a pharmaceutically acceptable salt thereof, or a pharmaceutical composition for use according to any one of clauses 1-5 and 22-26 in an individual afflicted with vitiligo, as characterized by a VASI score of at least 25%.
29. A compound, or a pharmaceutically acceptable salt thereof, or a pharmaceutical composition for use according to any one of clauses 1-5 and 22-26 in an individual afflicted with vitiligo, as characterized by a VASI score of at least 50%.
30. A compound, or a pharmaceutically acceptable salt thereof, or a pharmaceutical composition for use according to any one of clauses 1-5 and 22-26 in an individual afflicted with vitiligo, as characterized by a VASI score of at least 75%.
31. A compound, or a pharmaceutically acceptable salt thereof, or a pharmaceutical composition for use according to any one of clauses 1-Sand 22-26, wherein the VASI improvement is at least 10%, 20%, 30%, 40%, or 50% after treatment compared to before treatment.
32. A method for the treatment the prophylaxis and/or treatment of vitiligo comprising the steps of:
   measuring the depigmentation of the hands, upper extremities (excluding hands), trunk, lower extremities (excluding the feet), and feet of an individual using the hand as a unit (palm plus the volar surface of all the digits),
   calculating a VASI score by the formula:

$$VASI = {\sum }_{all body site} [ Hand Unit ] \times [ Residual Depigmentation ]$$

determining a daily dose of the compound according to Formula I, or a pharmaceutically acceptable salt thereof comprised between 25 mg and 400 mg for administration to said individual.
33. A compound, or a pharmaceutically acceptable salt thereof, or a pharmaceutical composition for use according to any one of clauses 1-5 and 22-26, wherein the administration of said compound, or a pharmaceutically acceptable salt thereof, or a pharmaceutical composition results in detectable changes of at least one vitiligo-associated biomarker.
34. A compound, or a pharmaceutically acceptable salt thereof, or a pharmaceutical composition for use according to any one of clauses 1-5 and 22-26 in an individual afflicted with vitiligo, said use comprising the steps of
   a) assaying a blood sample,
   b) measuring levels of at least one vitiligo-associated biomarker,
   c) administering a therapeutically effective amount of the compound, or a pharmaceutically acceptable salt thereof to the individual,
   d) measuring the post-treatment level of said vitiligo-associated biomarker and comparing with pre-treatment levels, and
   e) adjusting the dosage of the compound, or a pharmaceutically acceptable salt thereof to be administered to the individual, wherein said dosage is increased when a decrease of less than 2% after 12 weeks in levels of said vitiligo-associated biomarker is detected.
35. A compound, or a pharmaceutically acceptable salt thereof, for use according to clause 34, wherein the biomarker is IFN_{γ}.
36. A compound, or a pharmaceutically acceptable salt thereof, for use according to clause 35, wherein the biomarker level is decreased by at least 5%, at least 10%, or at least 15% compared to pre-treatment levels.
37. A compound, or a pharmaceutically acceptable salt thereof, or a pharmaceutical composition for use according to clause 35, wherein the IFN_{γ} are decreased by at least 5% compared to pre-treatment levels after 12 weeks of treatment.
38. A pharmaceutical composition for use in the prophylaxis and/or treatment of cutaneous lupus, comprising the compound according to Formula I, or a pharmaceutically acceptable salt thereof, and a pharmaceutically acceptable carrier, excipient, or diluent.
39. A pharmaceutical composition for use according to clause 38, comprising a further therapeutic agent.
40. The compound or a pharmaceutically acceptable salt thereof, for use according to clause 5 or the pharmaceutical composition for use according to clause 39, wherein the further therapeutic agent is an agent for the prophylaxis and/or treatment of cutaneous lupus.
41. The compound or a pharmaceutically acceptable salt thereof, for use according to clause 5 or the pharmaceutical composition for use according to clause 39, wherein further therapeutic agent is one or more of topical steroids, corticosteroids, calcineurin inhibitors, antimalarial drugs, retinoids, methotrexate, thalidomide, ciclosporin, dapsone, gold, clofazamine, cyclophosphamide, and immunoglobulin.
42. A compound, or a pharmaceutically acceptable salt thereof, for use according to any one of clauses 1-5, or a pharmaceutical composition according to clause 38 or 39 in an individual afflicted with cutaneous lupus.
43. A compound, or a pharmaceutically acceptable salt thereof, for use according to any one of clauses 1-5, or a pharmaceutical composition according for use according to clauses 38 or 39, wherein said cutaneous lupus is selected from the group consisting of chronic cutaneous lupus erythematosus, subacute cutaneous lupus erythematosus, discoid lupus erythematosus, acute cutaneous lupus erythematosus, and drug-induced lupus erythematosus.
44. A compound, or a pharmaceutically acceptable salt thereof, or a pharmaceutical composition for use according to any one of clauses 1-5 or 38-43, wherein the administration of said compound, or a pharmaceutically acceptable salt thereof, or a pharmaceutical composition results in detectable changes of a cutaneous lupus-associated biomarker.
45. A compound, or a pharmaceutically acceptable salt thereof, or a pharmaceutical composition for use according to any one of clauses 1-5 or 38-43 in an individual afflicted with cutaneous lupus, said use comprising the steps of
   a) assaying a blood sample,
   b) measuring levels of at least one cutaneous lupus-associated biomarker,
   c) administering a therapeutically effective amount of the compound, or a pharmaceutically acceptable salt thereof to the individual,
   d) measuring the post-treatment level of said cutaneous lupus-associated biomarker and comparing with pre-treatment levels, and
   e) adjusting the dosage of the compound, or a pharmaceutically acceptable salt thereof to be administered to the individual, wherein said dosage is increased when a decrease of less than 2% after 12 weeks in levels of said cutaneous lupus-associated biomarker is detected.
46. A compound, or a pharmaceutically acceptable salt thereof, or a pharmaceutical composition for use according to clause 44 or 45, wherein the biomarker is IL6 or complement component.
47. A compound, or a pharmaceutically acceptable salt thereof, or a pharmaceutical composition for use according to clause 44 or 45, wherein the biomarker is IL6.
48. A compound, or a pharmaceutically acceptable salt thereof, or a pharmaceutical composition for use, according to any one of clauses 45-47, wherein the biomarker level is decreased by at least 5%, at least 10%, at least 15%, at least 20%, at least 30%, at least 40%, at least 50%, at least 60%, at least 70%, at least 80%, at least 90% compared to pre-treatment levels.
49. A compound, or a pharmaceutically acceptable salt thereof, or a pharmaceutical composition according for use according to one of clauses 1-5 or 42-48, in an individual afflicted with cutaneous lupus as characterized by the Cutaneous Lupus Erythematosus Disease Area and Severity Index (CLASI).
50. A compound, or a pharmaceutically acceptable salt thereof, or a pharmaceutical composition for use according to any one of clauses 1-5 or 42-48 in an individual afflicted with cutaneous lupus, as characterized by a CLASI score of at least 5.
51. A compound, or a pharmaceutically acceptable salt thereof, or a pharmaceutical composition for use according to any one of clauses 1-5 or 42-48 in an individual afflicted with cutaneous lupus, as characterized by a CLASI score of at least 10.
52. A compound, or a pharmaceutically acceptable salt thereof, or a pharmaceutical composition for use according to any one of clauses 1-5 or 42-48 in an individual afflicted with cutaneous lupus, as characterized by a CLASI score of at least 15.
53. A compound, or a pharmaceutically acceptable salt thereof, or a pharmaceutical composition for use according to any one of clauses 1-5 or 42-48 in an individual afflicted with cutaneous lupus, as characterized by a CLASI score of at least 20.
54. A pharmaceutical composition for use in the prophylaxis and/or treatment of lupus nephritis, comprising the compound according to Formula I, or a pharmaceutically acceptable salt thereof, and a pharmaceutically acceptable carrier, excipient, or diluent.
55. A pharmaceutical composition for use according to clause 54, comprising a further therapeutic agent.
56. The compound or a pharmaceutically acceptable salt thereof, for use according to clause 5 or the pharmaceutical composition for use according to clause 55, wherein the further therapeutic agent is an agent for the prophylaxis and/or treatment of lupus nephritis.
57. The compound or a pharmaceutically acceptable salt thereof, for use according to clause 5 or the pharmaceutical composition for use according to clause 55, wherein further therapeutic agent is selected from angiotensin converting enzyme (ACE) inhibitors, angiotensin receptor blockers (ARBs), antimalarials, statins, cyclophosphamide, azathioprine, 6-mercaptopurine, abatacept, rituximab, belimumab, cyclosporine and other calcineurin inhibitors.
58. A compound, or a pharmaceutically acceptable salt thereof, for use according to any one of clauses 1-5, or a pharmaceutical composition according to clause 56 in an individual afflicted with lupus nephritis.
59. A compound, or a pharmaceutically acceptable salt thereof, for use according to any one of clauses 1-5, or a pharmaceutical composition according for use according to clauses 54 or 55, wherein said lupus nephritis is membranous lupus nephritis.
60. A compound, or a pharmaceutically acceptable salt thereof, for use according to any one of clauses 1-5, or a pharmaceutical composition according for use according to clauses 54 or 55, wherein the administration of said compound, or a pharmaceutically acceptable salt thereof, or a pharmaceutical composition results in detectable changes of a biomarker.
61. A compound, or a pharmaceutically acceptable salt thereof, or a pharmaceutical composition for use according to any one of clauses 1-5 or 54-59 in an individual afflicted with lupus nephritis, said use comprising the steps of:
   a) assaying a blood sample,
   b) measuring levels of at least one lupus nephritis-associated biomarker,
   c) administering a therapeutically effective amount of the compound, or a pharmaceutically acceptable salt thereof to the individual,
   d) measuring the post-treatment level of said lupus nephritis-associated biomarker and comparing with pre-treatment levels, and
   e) adjusting the dosage of the compound, or a pharmaceutically acceptable salt thereof to be administered to the individual, wherein said dosage is increased when a decrease of less than 2% after 12 weeks in levels of said lupus nephritis-associated biomarker is detected.
62. A compound, or a pharmaceutically acceptable salt thereof, or a pharmaceutical composition for use according to clause 60 or 61, wherein the biomarker is selected from the group consisting of IL-6, IL-10, IFNγ and TGFβ.
63. A compound, or a pharmaceutically acceptable salt thereof, or a pharmaceutical composition for use according to clause 61 or 62, wherein the biomarker is IL6.
64. A compound, or a pharmaceutically acceptable salt thereof, or a pharmaceutical composition for use according to clause 61 or 62, wherein the biomarker level is increased/decreased by at least 5%, at least 10%, at least 15%, at least 20%, at least 30%, at least 40%, at least 50%, at least 60%, at least 70%, at least 80%, at least 90% compared to normal level.
65. A compound, or a pharmaceutically acceptable salt thereof, for use according to any one of clauses 1-5, or a pharmaceutical composition according for use according to clauses 54 or 55, wherein the administration of said compound, or a pharmaceutically acceptable salt thereof, or said pharmaceutical composition reduces proteinuria in the subject.
66. A compound, or a pharmaceutically acceptable salt thereof, or a pharmaceutical composition according for use according to clause 65, wherein proteinuria reduction is measured by 24 hour urine protein, 24 hour protein to creatinine ratio, spot protein to creatinine ratio, 24 hour urine albumin, 24 hour albumin to creatinine ratio, spot albumin to creatinine ratio, or by a urinary dipstick.
67. A compound, or a pharmaceutically acceptable salt thereof, for use according to any one of clauses 1-5, or a pharmaceutical composition according for use according to clauses 54 or 55, wherein the periodic administration of said compound, or a pharmaceutically acceptable salt thereof reduces the subject's protein to creatinine ratio.
68. A compound, or a pharmaceutically acceptable salt thereof, or a pharmaceutical composition according for use according to clause 67, wherein the subject's protein to creatinine ratio is reduced by at least 50% as compared to baseline.
69. A pharmaceutical composition for use in the prophylaxis and/or treatment of sarcoïdosis and/or a sarcoïdosis-related condition, comprising the compound according to Formula I, or a pharmaceutically acceptable salt thereof, and a pharmaceutically acceptable carrier, excipient, or diluent.
70. A pharmaceutical composition for use according to clause 69, comprising a further therapeutic agent.
71. The compound or a pharmaceutically acceptable salt thereof, for use according to clause 5 or the pharmaceutical composition for use according to clause 69, wherein the further therapeutic agent is an agent for the prophylaxis and/or treatment of sarcoïdosis and/or a sarcoïdosis-related condition.
72. The compound or a pharmaceutically acceptable salt thereof, for use according to clause 5 or the pharmaceutical composition for use according to clause 69, wherein further therapeutic agent is one or more of corticosteroid, prednisone, methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, a tetracycline antibiotic.
73. A compound, or a pharmaceutically acceptable salt thereof, for use according to any one of clauses 1-5, or a pharmaceutical composition according to clause 69 or 70 in an individual afflicted with sarcoïdosis and/or a sarcoïdosis-related condition.
74. A compound, or a pharmaceutically acceptable salt thereof, for use according to any one of clauses 1-5 or a pharmaceutical composition according for use according to clauses 69 or 70, wherein said sarcoïdosis and/or a sarcoïdosis-related condition is selected from the group consisting of cardiac sarcoïdosis, cutaneous sarcoïdosis, hepatic sarcoïdosis, pulmonary sarcoïdosis, neurosarcoïdosis, Lofgren's syndrome, and chronic cutaneous sarcoïdosis.
75. A compound, or a pharmaceutically acceptable salt thereof, for use according to any one of clauses 1-5, or a pharmaceutical composition according for use according to clause 69 or 70, wherein the administration of said compound, or a pharmaceutically acceptable salt thereof, or a pharmaceutical composition results in detectable changes of a biomarker.
76. A compound, or a pharmaceutically acceptable salt thereof, for use according to any one of clauses 1-5, or a pharmaceutical composition according for use according to clause 69 or 70, in an individual afflicted with sarcoïdosis and/or a sarcoïdosis-related condition, said use comprising the steps of
   a) assaying a blood sample,
   b) measuring levels of at least one sarcoïdosis and/or a sarcoïdosis-related condition-associated biomarker,
   c) administering a therapeutically effective amount of the compound, or a pharmaceutically acceptable salt thereof to the individual,
   d) measuring the post-treatment level of said sarcoïdosis and/or a sarcoïdosis-related condition-associated biomarker and comparing with pre-treatment levels, and
   e) adjusting the dosage of compound, or a pharmaceutically acceptable salt thereof to be administered to the individual, wherein said dosage is increased when a decrease of less than 2% after 12 weeks in levels of said sarcoïdosis and/or a sarcoïdosis-related condition-associated biomarker is detected.
77. A compound, or a pharmaceutically acceptable salt thereof, or a pharmaceutical composition for use according to clause 75 or 76, wherein the biomarker is CXCL9 or CXCL10 expression in blood.
78. A compound, or a pharmaceutically acceptable salt thereof, or a pharmaceutical composition for use according to clause 75 or 76, wherein the biomarker is CXCL10 expression in blood.
79. A compound, or a pharmaceutically acceptable salt thereof, or a pharmaceutical composition for use, according to clause 75 or 76, wherein the biomarker level is decreased by at least 5%, at least 10%, at least 15%, at least 20%, at least 30%, at least 40%, at least 50%, at least 60%, at least 70%, at least 80%, at least 90% compared to pre-treatment levels.
80. A compound, or a pharmaceutically acceptable salt thereof, for use according to any one of clauses 1-5, or a pharmaceutical composition according for use according to clause 69 or 70, wherein the administration of said compound, or a pharmaceutically acceptable salt thereof, or said pharmaceutical composition causes a detectable improvement in one or more symptoms of said sarcoïdosis
81. A compound, or a pharmaceutically acceptable salt thereof, for use, or a pharmaceutical composition according for use according to clause 80, wherein said symptom is one or more of formation of granulomas, fatigue, weight loss, fever, aches, pains, arthritis, dry eyes, swelling of the knees, blurry vision, shortness of breath, cough and skin lesions.
82. A pharmaceutical composition for use in the prophylaxis and/or treatment of giant cell arteritis, comprising the compound according to Formula I, or a pharmaceutically acceptable salt thereof, and a pharmaceutically acceptable carrier, excipient, or diluent.
83. A pharmaceutical composition for use according to clause 6, comprising a further therapeutic agent.
84. The compound or a pharmaceutically acceptable salt thereof, according to clause 5 or the pharmaceutical composition for use according to clause 82, wherein the further therapeutic agent is an agent for the prophylaxis and/or treatment of giant cell arteritis.
85. A compound, or a pharmaceutically acceptable salt thereof, for use according to any one of clauses 1-5, or a pharmaceutical composition according to clause 82 or 83 in an individual afflicted with giant cell arteritis.
86. A compound, or a pharmaceutically acceptable salt thereof or a pharmaceutical composition, for use according to any one of clauses 1-5 or 82-85, in an individual afflicted with giant cell arteritis, as characterized by a CRP level greater than 3 mg/L.
87. A compound, or a pharmaceutically acceptable salt thereof or a pharmaceutical composition, for use according to any one of clauses 1-5 or 82-85, in an individual afflicted with giant cell arteritis, as characterized by an erythrocyte sedimentation rate greater than 30 mm/h
88. A method for the treatment the prophylaxis and/or treatment of giant cell arteritis comprising the steps of:
   measuring the ESR of an individual,
   measuring the CRP level of an individual,
   comparing said ESR and/or CRP level with normal values,
   determining a daily dose of the compound according to Formula I, or a pharmaceutically acceptable salt thereof comprised between 25 mg and 400 mg for administration to said individual.
89. A compound, or a pharmaceutically acceptable salt thereof according to any one of clauses 1-5, or a pharmaceutical composition for use according to clause 82 or 83, in an individual afflicted with giant cell arteritis, said use comprising the steps of
   a) assaying a blood sample,
   b) measuring levels of at least one giant cell arteritis-associated biomarker,
   c) administering a therapeutically effective amount of the compound, or a pharmaceutically acceptable salt thereof to the individual,
   d) measuring the post-treatment level of said giant cell arteritis-associated biomarker and comparing with pre-treatment levels, and
   e) adjusting the dosage of the compound, or a pharmaceutically acceptable salt thereof to be administered to the individual, wherein said dosage is increased when a decrease of less than 2% after 12 weeks in levels of said giant cell arteritis-associated biomarker is detected.
90. A compound, or a pharmaceutically acceptable salt thereof, for use according to clause 89, wherein the biomarker is IL6.
91. A compound, or a pharmaceutically acceptable salt thereof, for use according to clause 89, wherein the biomarker is ILL
92. A compound, or a pharmaceutically acceptable salt thereof, for use according to clause 89, wherein the biomarker is GM-CSF.
93. A compound, or a pharmaceutically acceptable salt thereof, for use according to any one of clauses 89-92, wherein the biomarker level is decreased by at least 1.5%, at least 5%, at least 10%, at 15% compared to pre-treatment level.
94. A compound, or a pharmaceutically acceptable salt thereof, for use according to any one of clauses 89-92, wherein the biomarker levels are decreased by at least 1.5%, at least 2%, at least 5%, at least 10% after 12 weeks.
95. A compound, or a pharmaceutically acceptable salt thereof according to clauses 1-5, or a pharmaceutical composition for use according to any one of clauses 6, 7, 22, 23, 38, 39, 54, 55, 69, 70, 82, and 83, wherein the compound is administered at a dose of 100 mg b.i.d.
96. A compound, or a pharmaceutically acceptable salt thereof according to clauses 1-5, or a pharmaceutical composition for use according to any one of clauses 6, 7, 22, 23, 38, 39, 54, 55, 69, 70, 82, and 83, wherein the compound is administered at a dose of 200 mg qd.
97. A compound, or a pharmaceutically acceptable salt thereof, or a pharmaceutical composition for use according to clause 95 or 96 wherein the compound, or a pharmaceutically acceptable salt thereof, or a pharmaceutical composition are administered over a period of least 4, 6, 8, 10, 12, 14, 16, 20, or 24 weeks.
98. A compound, or a pharmaceutically acceptable salt thereof, or a pharmaceutical composition for use according to clause 95 or 96, wherein the compound, or a pharmaceutically acceptable salt thereof, or a pharmaceutical composition are administered over a period of least 12 weeks.

### Pharmaceutical Compositions

When employed as a pharmaceutical, a compound of the invention is typically administered in a pharmaceutical composition. Such compositions can be prepared in a manner well known in the pharmaceutical art and comprise a compound of the invention, e.g. a compound according to Formula (I). Generally, a compound of the invention is administered in an effective amount. The amount of a compound of the invention actually administered will typically be determined by a physician, in the light of the relevant circumstances, including the condition to be treated, the chosen route of administration, the actual compound of the invention administered, the age, weight, and response of the individual patient, the severity of the patient's symptoms, and the like.

The pharmaceutical compositions of this invention can be administered by a variety of routes including oral, rectal, transdermal, subcutaneous, intra-articular, intravenous, intramuscular, and intranasal. Depending on the intended route of delivery, a compound of the invention is preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term ‘unit dosage forms’ refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions. In such compositions, the compound of the invention according to Formula I is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or a compound of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint or orange flavoring.

Injectable compositions are typically based upon injectable sterile saline or phosphate-buffered saline or other injectable carriers known in the art. As before, the compound of the invention according to Formula (I) in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

A compound of the invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17^{th} edition, 1985, Mack Publishing Company, Easton, Pa., which is incorporated herein by reference.

A compound of the invention can also be administered in sustained release forms or from sustained release drug delivery systems. A description of representative sustained release materials can be found in Remington's Pharmaceutical Sciences.

The following formulation examples illustrate representative pharmaceutical compositions that may be prepared in accordance with this invention. The present invention, however, is not limited to the following pharmaceutical compositions.

### Formulation 1—Tablets

A compound of the invention according to Formula I may be admixed as a dry powder with a dry gelatin binder in an approximate 1:2 weight ratio. A minor amount of magnesium stearate may be added as a lubricant. The mixture may be formed into 240-270 mg tablets (80-90 mg of active compound of the invention according to Formula I per tablet) in a tablet press.

### Formulation 2—Capsules

A compound of the invention according to Formula I may be admixed as a dry powder with a starch diluent in an approximate 1:1 weight ratio. The mixture may be filled into 250 mg capsules (125 mg of active compound of the invention according to Formula I per capsule).

### Formulation 3—Liquid

A compound of the invention according to Formula I (125 mg), may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color may be diluted with water and added with stirring. Sufficient water may then be added with stirring. Further sufficient water may be then added to produce a total volume of 5 mL.

### Formulation 4—Tablets

A compound of the invention according to Formula I may be admixed as a dry powder with a dry gelatin binder in an approximate 1:2 weight ratio. A minor amount of magnesium stearate may be added as a lubricant. The mixture may be formed into 450-900 mg tablets (150-300 mg of active compound of the invention according to Formula I) in a tablet press.

### Formulation 5—Injection

A compound of the invention according to Formula I may be dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of A compound of the invention according to Formula I (50 g) methylparaben (0.25 g), propylparaben (0.15 g), sodium lauryl sulfate (10 g), and propylene glycol (120 g) dissolved in water (about 370 g) may be added and the resulting mixture may be stirred until it congeals.

### Methods of Treatment

In one embodiment, the present invention provides a compound of the invention, or pharmaceutical compositions comprising a compound of the invention, for use in medicine. In a particular embodiment, the present invention provides a compound of the invention or pharmaceutical compositions comprising a compound of the invention, for use in the prophylaxis and/or treatment of alopecia areata.

In one embodiment, the present invention provides a compound of the invention and another therapeutic agent or pharmaceutical compositions comprising a compound of the invention and another therapeutic agent, for use in the prophylaxis and/or treatment of alopecia areata. In a particular embodiment, said another therapeutic agent is an alopecia areata treatment agent. In a particular embodiment said another agent is selected from steroids (such as clobetasol propionate, desoximetasone, hydrocortisone, methylprednisolone, prednisone, prednisolone, budesonide, or dexamethasone), topical immunotherapy (Dinitrochlorobenzene, squaric acid dibutylester (SADBE), diphenylcyclopropenone (DPCP)), topical minoxidil, anthralin, psoralen and immunosuppressants (eg, cyclosporin, azathioprine, methotrexate).

In another embodiment, the present invention provides compounds of the invention, or pharmaceutical compositions comprising a compound of the invention for use in the manufacture of a medicament for use in the prophylaxis and/or treatment of alopecia areata.

In one embodiment, the present invention provides a compound of the invention and another therapeutic agent, or pharmaceutical compositions comprising a compound of the invention and another therapeutic agent for use in the manufacture of a medicament for use in the prophylaxis and/or treatment of alopecia areata. In a particular embodiment, said another therapeutic agent is an alopecia areata treatment agent. In a particular embodiment said another agent is selected from steroids (such as clobetasol propionate, desoximetasone, hydrocortisone, methylprednisolone, prednisone, prednisolone, budesonide, or dexamethasone), topical immunotherapy (Dinitrochlorobenzene, squaric acid dibutylester (SADBE), diphenylcyclopropenone (DPCP)), topical minoxidil, anthralin, psoralen and immunosuppressants (eg, cyclosporin, azathioprine, methotrexate).

In additional method of treatment aspects, this invention provides methods of prophylaxis and/or treatment of a mammal afflicted with alopecia areata, which methods comprise the administration of an effective amount of a compound of the invention or one or more of the pharmaceutical compositions herein described for the treatment or prophylaxis of said condition.

In one embodiment, the present invention provides methods of prophylaxis and/or treatment of a mammal afflicted with alopecia areata, wherein said methods comprise an administration of another therapeutic agent with a compound of the invention. In a particular embodiment, said another therapeutic agent is an alopecia areata treatment agent. In a particular embodiment said another agent is selected from steroids (such as clobetasol propionate, desoximetasone, hydrocortisone, methylprednisolone, prednisone, prednisolone, budesonide, or dexamethasone), topical immunotherapy (Dinitrochlorobenzene, squaric acid dibutylester (SADBE), diphenylcyclopropenone (DPCP)), topical minoxidil, anthralin, psoralen and immunosuppressants (eg, cyclosporin, azathioprine, methotrexate).

In one embodiment, the present invention provides a compound of the invention, or pharmaceutical compositions comprising a compound of the invention, for use in medicine. In a particular embodiment, the present invention provides a compound of the invention or pharmaceutical compositions comprising a compound of the invention, for use in the prophylaxis and/or treatment of vitiligo.

In one embodiment, the present invention provides a compound of the invention and another therapeutic agent or pharmaceutical compositions comprising a compound of the invention and another therapeutic agent, for use in the prophylaxis and/or treatment of vitiligo. In a particular embodiment, said another therapeutic agent is an vitiligo treatment agent. In a particular embodiment said another agent is selected from topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), argan oil and retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™ Enbrel™, Humira™, Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In another embodiment, the present invention provides compounds of the invention, or pharmaceutical compositions comprising a compound of the invention for use in the manufacture of a medicament for use in the prophylaxis and/or treatment of vitiligo.

In one embodiment, the present invention provides a compound of the invention and another therapeutic agent, or pharmaceutical compositions comprising a compound of the invention and another therapeutic agent for use in the manufacture of a medicament for use in the prophylaxis and/or treatment of vitiligo. In a particular embodiment, said another therapeutic agent is a vitiligo treatment agent. In a particular embodiment said another agent is selected from topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), argan oil and retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™, Enbrel™, Humira™, Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In additional method of treatment aspects, this invention provides methods of prophylaxis and/or treatment of a mammal afflicted with vitiligo, which methods comprise the administration of an effective amount of a compound of the invention or one or more of the pharmaceutical compositions herein described for the treatment or prophylaxis of said condition.

In one embodiment, the present invention provides methods of prophylaxis and/or treatment of a mammal afflicted with vitiligo, wherein said methods comprise administration of another therapeutic agent with a compound of the invention. In a particular embodiment, said another therapeutic agent is a vitiligo treatment agent. In a particular embodiment said another agent is selected from topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), argan oil and retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™, Enbrel™, Humira™ Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In one embodiment, the compound of the invention is co-administered with another agent for the treatment and/or prophylaxis of vitiligo, particular agents include but are not limited to: topical treatments such as bath solutions, moisturizers, medicated creams and ointments containing coal tar, dithranol (anthralin), corticosteroids like desoximetasone (Topicort™), fluocinonide, vitamin D3 analogues (for example, calcipotriol), argan oil and retinoids (etretinate, acitretin, tazarotene), systemic treatments such as methotrexate, cyclosporine, retinoids, tioguanine, hydroxyurea, sulfasalazine, mycophenolate mofetil, azathioprine, tacrolimus, fumaric acid esters or biologics such as Amevive™, Enbrel™, Humira™ Remicade™, Raptiva™ and ustekinumab (a IL-12 and IL-23 blocker). Additionally, a compound of the invention may be administered in combination with other therapies including, but not limited to phototherapy, or photochemotherapy (e.g. psoralen and ultraviolet A phototherapy (PUVA)).

In one embodiment, the present invention provides compounds of the invention, or pharmaceutical compositions comprising a compound of the invention, for use in medicine. In a particular embodiment, the present invention provides a compound of the invention or pharmaceutical compositions comprising a compound of the invention, for use in the prophylaxis and/or treatment of cutaneous lupus.

In one embodiment, the present invention provides a compound of the invention and another therapeutic agent or pharmaceutical compositions comprising a compound of the invention and another therapeutic agent, for use in the prophylaxis and/or treatment of cutaneous lupus. In a particular embodiment, said another therapeutic agent is a cutaneous lupus treatment agent. In a particular embodiment said another agent is selected from the group consisting of topical steroids, corticosteroids, calcineurin inhibitors, antimalarial drugs, retinoids, methotrexate, thalidomide, ciclosporin, dapsone, gold compounds, clofazamine, cyclophosphamide, and immunoglobulin.

The present invention provides the compound of the invention or pharmaceutical compositions comprising the compound of the invention, for use in the prophylaxis and/or treatment of cutaneous lupus, wherein said cutaneous lupus includes, but is not limited to, cutaneous lupus erythematosus (CLE), acute cutaneous lupus erythematosus (ACLE), subacute cutaneous lupus erythematosus (SCLE), chronic cutaneous lupus erythematosus (CCLE) or discoid lupus erythematosus (DLE).

In some embodiments, the present invention provides the compound of the invention or pharmaceutical compositions comprising the compound of the invention, for use in the prophylaxis and/or treatment of ACLE.

In further embodiments, the present invention provides the compound of the invention or pharmaceutical compositions comprising the compound of the invention, for use in the prophylaxis and/or treatment of SCLE.

In further embodiments, the present invention provides the compound of the invention or pharmaceutical compositions comprising the compound of the invention, for use in the prophylaxis and/or treatment of CCLE or DLE. In another embodiment, the present invention provides the compound of the invention, or pharmaceutical compositions comprising the compound of the invention for use in the manufacture of a medicament for use in the prophylaxis and/or treatment of cutaneous lupus.

In one embodiment, the present invention provides a compound of the invention and another therapeutic agent, or pharmaceutical compositions comprising a compound of the invention and another therapeutic agent for use in the manufacture of a medicament for use in the prophylaxis and/or treatment of cutaneous lupus. In a particular embodiment, said another therapeutic agent is a cutaneous lupus treatment agent. In a particular embodiment said another agent is selected from the group consisting of topical steroids, corticosteroids, calcineurin inhibitors, antimalarial drugs, retinoids, methotrexate, thalidomide, ciclosporin, dapsone, gold compounds, clofazamine, cyclophosphamide, and immunoglobulin.

The present invention provides the compound of the invention, or pharmaceutical compositions comprising the compound of the invention for use in the manufacture of a medicament for use in the prophylaxis and/or treatment of cutaneous lupus, wherein said cutaneous lupus includes, but is not limited to, cutaneous lupus erythematosus (CLE), acute cutaneous lupus erythematosus (ACLE), subacute cutaneous lupus erythematosus (SCLE), chronic cutaneous lupus erythematosus (CCLE) or discoid lupus erythematosus (DLE).

In some embodiments, the present invention provides the compound of the invention, or pharmaceutical compositions comprising the compound of the invention for use in the manufacture of a medicament for use in the prophylaxis and/or treatment of ACLE.

In further embodiments, the present invention provides the compound of the invention, or pharmaceutical compositions comprising the compound of the invention for use in the manufacture of a medicament for use in the prophylaxis and/or treatment of SCLE.

In further embodiments, the present invention provides the compound of the invention, or pharmaceutical compositions comprising the compound of the invention for use in the manufacture of a medicament for use in the prophylaxis and/or treatment of CCLE or DLE.

In additional method of treatment aspects, this invention provides methods of prophylaxis and/or treatment of a mammal afflicted with cutaneous lupus, which methods comprise the administration of an effective amount of the compound of the invention or one or more of the pharmaceutical compositions described herein for the treatment or prophylaxis of said condition.

In one embodiment, the present invention provides methods of prophylaxis and/or treatment of a mammal afflicted with cutaneous lupus, wherein said methods comprise administration of another therapeutic agent with a compound of the invention. In a particular embodiment, the other therapeutic agent is a cutaneous lupus treatment agent. In a particular embodiment said another agent is selected from the group consisting of topical steroids, corticosteroids, calcineurin inhibitors, antimalarial drugs, retinoids, methotrexate, thalidomide, ciclosporin, dapsone, gold compounds, clofazamine, cyclophosphamide, and immunoglobulin.

The present invention provides methods of prophylaxis and/or treatment of a mammal afflicted with cutaneous lupus, wherein said cutaneous lupus includes, but is not limited to, cutaneous lupus erythematosus (CLE), acute cutaneous lupus erythematosus (ACLE), subacute cutaneous lupus erythematosus (SCLE), chronic cutaneous lupus erythematosus (CCLE) or discoid lupus erythematosus (DLE).

In some embodiments, the present invention provides methods of prophylaxis and/or treatment of ACLE.

In further embodiments, the present invention provides methods of prophylaxis and/or treatment of SCLE.

In further embodiments, the present invention provides methods of prophylaxis and/or treatment of CCLE or DLE.

In one embodiment, the present invention provides the compound of the invention, or pharmaceutical compositions comprising the compound of the invention, for use in medicine. In a particular embodiment, the present invention provides compounds of the invention or pharmaceutical compositions comprising a compound of the invention, for use in the prophylaxis and/or treatment of lupus nephritis.

In one embodiment, the present invention provides a compound of the invention and another therapeutic agent, or pharmaceutical compositions comprising a compound of the invention and another therapeutic agent for use in the prophylaxis and/or treatment of lupus nephritis. In a particular embodiment, the other therapeutic agent is a lupus nephritis treatment agent.

In a particular embodiment said another therapeutic agent is one or more of angiotensin converting enzyme (ACE) inhibitors, angiotensin receptor blockers (ARBs), antimalarials, statins, cyclophosphamide, azathioprine, 6-mercaptopurine, abatacept, rituximab, belimumab, cyclosporine or other calcineurin inhibitors.

In another embodiment, the present invention provides a compound of the invention, or pharmaceutical compositions comprising a compound of the invention for use in the manufacture of a medicament for use in the prophylaxis and/or treatment of lupus nephritis.

In one embodiment, the present invention provides the compound of the invention, or pharmaceutical compositions comprising a compound of the invention for use in the manufacture of a medicament for use in the prophylaxis and/or treatment of lupus nephritis. In a particular embodiment, the other therapeutic agent is a lupus nephritis treatment agent.

In a particular embodiment said another therapeutic agent is one or more of angiotensin converting enzyme (ACE) inhibitors, angiotensin receptor blockers (ARBs), antimalarials, statins, cyclophosphamide, azathioprine, 6-mercaptopurine, abatacept, rituximab, belimumab, cyclosporine or other calcineurin inhibitors.

In additional method of treatment aspects, this invention provides methods of prophylaxis and/or treatment of a mammal afflicted with lupus nephritis, which methods comprise the administration of an effective amount of the compound of the invention or one or more of the pharmaceutical compositions herein described for the treatment or prophylaxis of said condition.

In one embodiment, said methods of prophylaxis and/or treatment of a mammal afflicted with lupus nephritis comprise the administration of another therapeutic agent with a compound of the invention. In a particular embodiment, the other therapeutic agent is a lupus nephritis treatment agent.

In a particular embodiment said another therapeutic agent is one or more of angiotensin converting enzyme (ACE) inhibitors, angiotensin receptor blockers (ARBs), antimalarials, statins, cyclophosphamide, azathioprine, 6-mercaptopurine, abatacept, rituximab, belimumab, cyclosporine or other calcineurin inhibitors.

In a particular embodiment, the lupus nephritis to be treated or prevented is membranous lupus nephritis.

In a particular embodiment the administration of a compound of the invention reduces proteinuria in the subject.

In a particular embodiment the administration of a compound of the invention leads to proteinuria reduction as measured by 24 hour urine protein, 24 hour protein to creatinine ratio, spot protein to creatinine ratio, 24 hour urine albumin, 24 hour albumin to creatinine ratio, spot albumin to creatinine ratio, or by a urinary dipstick.

In a particular embodiment periodic administration of a compound of the invention reduces the subject's protein to creatinine ratio. More particularly the subject's protein to creatinine ratio is reduced by at least 50% as compared to pre-treatment levels.

In a particular embodiment a compound of the invention demonstrates clinically significant improvement of renal function during the induction phase, more specifically as measured by an improvement of glomerular filtration rate (GFR). Alternatively, the improvement is measured using the reduction of renal injury, primarily protein excretion and detection in active urinary sediment. In a particular embodiment, said improvement is measured using the levels of proteinuria. More particularly the proteinuria is reduced to below 0.5 g per 24-h urine collection sample.

In a particular embodiment a compound of the invention prevents renal flares.

In a particular embodiment the compounds of the invention or the pharmaceutical compositions as disclosed herein slow progression of chronic kidney disease (CKD).

In one embodiment, the present invention provides a compound of the invention, or pharmaceutical compositions comprising the compound of the invention, for use in medicine. In a particular embodiment, the present invention provides a compound of the invention or pharmaceutical compositions comprising a compound of the invention, for use in the prophylaxis and/or treatment of sarcoïdosis and/or a sarcoïdosis-related condition.

In one embodiment, the present invention provides a compound of the invention and another therapeutic agent or pharmaceutical compositions comprising a compound of the invention and another therapeutic agent, for use in the prophylaxis and/or treatment of sarcoïdosis and/or a sarcoïdosis-related condition. In a particular embodiment, the other therapeutic agent is a sarcoïdosis treatment agent. In a particular embodiment said another therapeutic agent is an anti-inflammatory agent, a steroid, an immunosuppressant compound, or an antibiotic. In a specific embodiment said another therapeutic agent is a corticosteroid, prednisone, methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, a tetracycline antibiotic, or a combination thereof.

In a particular embodiment said sarcoïdosis is selected from the group consisting of cardiac sarcoïdosis, cutaneous sarcoïdosis, hepatic sarcoïdosis, pulmonary sarcoïdosis, neurosarcoïdosis, Lofgren's syndrome, and chronic cutaneous sarcoïdosis.

In another embodiment, the present invention provides a compound of the invention, or pharmaceutical compositions comprising a compound of the invention for use in the manufacture of a medicament for use in the prophylaxis and/or treatment of sarcoïdosis and/or a sarcoïdosis-related condition.

In one embodiment, the present invention provides a compound of the invention and another therapeutic agent, or pharmaceutical compositions comprising a compound of the invention and another therapeutic agent for use in the manufacture of a medicament for use in the prophylaxis and/or treatment of sarcoïdosis and/or a sarcoïdosis-related condition. In a particular embodiment, the other therapeutic agent is a sarcoïdosis treatment agent. In a particular embodiment said another therapeutic agent is an anti-inflammatory agent, a steroid, an immunosuppressant compound, or an antibiotic.

In a specific embodiment said another therapeutic agent is a corticosteroid, prednisone, methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, a tetracycline antibiotic, or a combination thereof

In a particular embodiment said sarcoïdosis is selected from the group consisting of cardiac sarcoïdosis, cutaneous sarcoïdosis, hepatic sarcoïdosis, pulmonary sarcoïdosis, neurosarcoïdosis, Lofgren's syndrome, and chronic cutaneous sarcoïdosis

In additional method of treatment aspects, this invention provides methods of prophylaxis and/or treatment of a mammal afflicted with sarcoïdosis and/or a sarcoïdosis-related condition, which methods comprise the administration of an effective amount of a compound of the invention or one or more of the pharmaceutical compositions herein described for the treatment or prophylaxis of said condition.

In one embodiment, the present invention provides pharmaceutical compositions comprising a compound of the invention, and another therapeutic agent. In a particular embodiment, the other therapeutic agent is a sarcoïdosis treatment agent. In a particular embodiment said another therapeutic agent is an anti-inflammatory agent, a steroid, an immunosuppressant compound, or an antibiotic. In a specific embodiment said another therapeutic agent is a corticosteroid, prednisone, methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, a tetracycline antibiotic, or a combination thereof.

In a particular embodiment said sarcoïdosis is selected from the group consisting of cardiac sarcoïdosis, cutaneous sarcoïdosis, hepatic sarcoïdosis, pulmonary sarcoïdosis, neurosarcoïdosis, Lofgren's syndrome, and chronic cutaneous sarcoïdosis

In a particular embodiment the therapeutically effective amount is an amount sufficient to cause a detectable improvement in one or more symptoms of sarcoïdosis. In a more particular embodiment the administration of the compound, or a pharmaceutically acceptable salt thereof, or the pharmaceutical compositions as disclosed here demonstrate a detectable improvement in one or more symptoms of sarcoïdosis

In a particular embodiment said symptom is one or more selected from the group consisting of formation of granulomas, fatigue, weight loss, fever, aches, pains, arthritis, dry eyes, swelling of the knees, blurry vision, shortness of breath, cough and skin lesions.

In a particular embodiment the administration of a compound of the invention, or a pharmaceutical composition as disclosed here leads to downregulation of CXCL9 and CXCL10 expression in blood.

In one embodiment, the present invention provides compounds of the invention, or pharmaceutical compositions comprising a compound of the invention, for use in medicine. In a particular embodiment, the present invention provides compounds of the invention or pharmaceutical compositions comprising a compound of the invention, for use in the prophylaxis and/or treatment of giant cell arteritis.

In another embodiment, the present invention provides compounds of the invention, or pharmaceutical compositions comprising a compound of the invention for use in the manufacture of a medicament for use in the prophylaxis and/or treatment of giant cell arteritis.

In additional method of treatment aspects, this invention provides methods of prophylaxis and/or treatment of a mammal afflicted with giant cell arteritis, which methods comprise the administration of an effective amount of a compound of the invention or one or more of the pharmaceutical compositions herein described for the treatment or prophylaxis of said condition.

In one embodiment, the present invention provides pharmaceutical compositions comprising a compound of the invention, and another therapeutic agent. In a particular embodiment, the other therapeutic agent is a giant cell arteritis treatment agent.

In one embodiment, a compound of the invention is co-administered with another agent for the treatment and/or prophylaxis of GCA, particular agents include steroids (such as hydrocortisone, methylprednisolone, prednisone, prednisolone, budesonide, or dexamethasone), aspirin, immunosuppressant agents (eg, cyclosporin, azathioprine, methotrexate), dapsone, cyclophosphamide, and/or biological DMARDS (for example but without limitation infliximab, etanercept, adalimumab, rituximab, and abatacept).

Injection dose levels range from about 0.1 mg/kg/h to at least 10 mg/kg/h, all for from about 1 to about 120 h and especially 24 to 96 h. A preloading bolus of from about 0.1 mg/kg to about 10 mg/kg or more may also be administered to achieve adequate steady state levels. The maximum total dose is not expected to exceed about 1 g/day for a 40 to 80 kg human patient.

For the prophylaxis and/or treatment of long-term conditions, such as degenerative conditions, the regimen for treatment usually stretches over many months or years so oral dosing is preferred for patient convenience and tolerance. With oral dosing, one to four (1-4) regular doses daily, especially one to three (1-3) regular doses daily, typically one to two (1-2) regular doses daily, and most typically one (1) regular dose daily are representative regimens. Alternatively for long lasting effect drugs, with oral dosing, once every other week, once weekly, and once a day are representative regimens. In particular, dosage regimen can be every 1-14 days, more particularly 1-10 days, even more particularly 1-7 days, and most particularly 1-3 days.

Using these dosing patterns, each dose provides from about 1 to about 1000 mg of the compound of the invention, with particular doses each providing from about 10 to about 500 mg and especially about 30 to about 250 mg.

Transdermal doses are generally selected to provide similar or lower blood levels than are achieved using injection doses.

When used to prevent the onset of a condition, a compound of the invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of the invention can be administered as the sole active agent or it can be administered in combination with other therapeutic agents, including other compounds of the invention that demonstrate the same or a similar therapeutic activity and that are determined to be safe and efficacious for such combined administration. In a specific embodiment, co-administration of two (or more) agents allows for significantly lower doses of each to be used, thereby reducing the side effects seen.

In one embodiment, a compound of the invention or a pharmaceutical composition comprising a compound of the invention is administered as a medicament. In a specific embodiment, said pharmaceutical composition additionally comprises a further active ingredient.

By co-administration is included any means of delivering two or more therapeutic agents to the patient as part of the same treatment regimen, as will be apparent to the skilled person. Whilst the two or more agents may be administered simultaneously in a single formulation, i.e. as a single pharmaceutical composition, this is not essential. The agents may be administered in different formulations and at different times.

### Chemical Synthesis and Biological Profile

The compound of the invention according to Formula (I) has been extensively profiled, and data are disclosed in WO 2010/149769 (Menet and Smits, 2010). The synthesis of the salt and suitable formulations have been described in WO2015/117980, and in WO2015/117981.

Similarly, the compound of the invention according to Formula (II) has been extensively profiled, and data are disclosed in WO 2013/189771 (Van't Klooster et al., 2013).

### CLINICAL EXAMPLES

The compound according to Formula (I) has been administered to human in clinical trials and resulting data are disclosed in WO2016/165952 and WO2016/165953.

TABLE I
List of abbreviations used herein:

| Abbreviation | Definition |
|---|---|
| μL | microliter |
| μM | micromolar |
| ACLE | acute cutaneous lupus erythematosus |
| b.i.d. | bis in die (twice daily) |
| ACEi | angiotensin-converting enzyme inhibitor |
| ARB | angiotensin II receptor blocker |
| CCLE | chronic cutaneous lupus erythematosus |
| CFB | Change from baseline |
| CLASI | Cutaneous Lupus Erythematosus |
| | Disease Area and Severity Index |
| CLE | cutaneous lupus erythematosus |
| CRP | c-Reactive Protein |
| DBP | diastolic blood pressure |
| DMARD | disease-modifying anti-rheumatic drugs |
| ESRD | End stage renal disease |
| g | gram |
| h | hour |
| LOCF | last-observation-carried-forward |
| MTX | methotrexate |
| NRI | nonresponder imputation |
| NSAIDs | nonsteroidal anti-inflammatory drugs |
| q.d. | quo die (once a day) |
| RA | Rheumatoid arthritis |
| RNA | Ribonucleic acid |
| s | singlet |
| SALT | Severity alopecia tool |
| SCLE | subacute cutaneous lupus erythematosus |
| SBP | systolic blood pressure |
| shRNA | short hairpin RNA |
| SJC66 | swollen joint count |
| SLE | systemic lupus erythematosus |
| TJC68 | tender joint count |
| UV | ultraviolet |
| VASI | Vitiligo Area Scoring Index |

### Example 1. Biomarker Study

1.1. Study 1

1.1.1. Study Design

Double-blind, placebo-controlled add on study in subjects with moderately to severely active RA who have an inadequate response to MTX (oral or parenteral).

595 subjects randomized to one of 6 dose regimens of Compound 1 (dosed as a [Compound 1:HCl:3H₂O]) (3 dose levels administered either once or twice daily) or placebo on top of each subject's stable dose of MTX.

1.1.2. Study Duration

Treatment duration: 24 weeks.

1.1.3. Treatment

Compound 1 (dosed as a [Compound 1:HCl:3H₂O]) is dosed for twelve weeks once daily (q.d.) (50 mg, 100 mg or 200 mg) or twice daily (b.i.d.) (25 mg, 50 mg or 100 mg); or placebo.

At Week 12, the subjects on placebo who have not achieved 20% improvement in swollen joint count (SJC66) and tender joint count (TJC68) are re-randomized automatically to receive Compound 1 (dosed as a [Compound 1:HCl:3H₂O]) either at 100 mg q.d. or 50 mg b.i.d. doses in a blinded fashion; subjects on 50 mg q.d. who have not achieved 20% improvement in SJC66 and TJC68 will be assigned to 100 mg q.d. and subjects on 25 mg b.i.d. that have not achieved a 20% improvement in SJC66 and TJC68 will be assigned to 50 mg b.i.d. Subjects who switch treatment at week 12 are handled as if they discontinued at Week 12 for the purpose of statistical analysis, whereas subjects in the other groups will maintain their randomized treatment until Week 24.

1.1.4. Participants

1.1.4.1. Main Inclusion Criteria:

male or female subjects who are ≥18 years of age, on the day of signing informed consent,

diagnosis of RA at least 6 months prior to screening and meeting the 2010 ACR/EULAR criteria of RA and ACR functional class I-III (Aletaha et al., 2010),

≥6 swollen joints (from a 66 joint count) and ≥8 tender joints (from a 68 joint count) at Screening and at Baseline,

screening serum c-reactive protein (CRP)≥0.7×upper limit of laboratory normal range (ULN),

on MTX for ≥6 months and on a stable dose (15 to 25 mg/week) of MTX for at least 4 weeks prior to Screening and continued on their current regimen for the duration of the study. Stable doses of MTX as low as 10 mg/week are allowed when there is documented evidence of intolerance or safety issues at higher doses.

1.1.4.2. Main Exclusion Criteria:

current therapy with any disease-modifying anti-rheumatic drugs (DMARD) other than MTX, including oral or injectable gold, sulfasalazine, antimalarials, azathioprine, or D-penicillamine within 4 weeks prior to Baseline, cyclosporine within 8 weeks prior to Baseline, and leflunomide within 3 months prior to Baseline or a minimum 4 weeks prior to Baseline if after 11 days of standard cholestyramine therapy,

current or previous RA treatment with a biologic DMARD, with the exception of biologic DMARDs administered in a single clinical study setting more than 6 months prior to Screening (12 months for rituximab or other B cell depleting agents), where the biologic DMARD was effective, and if discontinued, this should not be due to lack of efficacy,

previous treatment at any time with a cytotoxic agent, other than MTX, before Screening.

1.1.5. Data Collection

1.1.5.1. Principle

The analysis was performed at Platine Pharma Services (Batiment Domilyon, Centre d'Infectiologie, 321 avenue Jean Jaurès, 69007 Lyon FRANCE).

18 parameters from human serum samples (GM-CSF, IFNγ, IL-1β, IL-2, IL-4, IL-5, IL-6, IL-7, IL-8, IL-10, IL-12 (p70), IL-13, IL-17A, IL-21, IL-23, MIP-1α, MIP-1β and TNF-α) are analyzed on a Luminex Plateform (BioPlex® L200) using a Luminex 18-Plex Kit (Millipore: HSTCMAG-28SK).

The Bio-Plex® multiplexing system is obtained by mixing different dyed magnetic beads coupled with antibodies specific for various analytes.

Coupled beads react with the sample containing the biomarker of interest. After a series of washes to remove unbound proteins, a biotinylated human cytokine detection antibody is added that generates a sandwich complex revealed by adding streptavidin-phycoerythrin (SA-PE) conjugates. Phycoerythrin (PE) serves as a fluorescent indicator.

The plate is loaded into the Bio-Plex reader and analytes present in the biological fluid are identified and quantified based on bead color-code (red laser 635 nm) and PE fluorescence (green laser 532 nm).

The concentration of bead bound analyte is proportional to the MFI (Median Fluorescent Intensity) and back-calculated from interpolation with the calibration curve fitted with a 4 or 5 parameters logistic (4PL or 5PL) regression model selected during method pre-validation.

1.2. Results

Treatment effect was assessed using an ANCOVA model on the changes from baseline, with factors: treatment, baseline value of the PD marker, geographical region and previous use of biologics.

Legend for pairwise comparisons versus placebo: +: p<0.10; *: p<0.05; **: p<0.01; ***: p<0.001

Performing the protocol described above afforded the results described below.

TABLE II
Week 4 observed-case table

| | Treatment | Mean (SE) change from baseline at Week 4, in pg/mL | | | | | | | | | |
|---|---|---|---|---|---|---|---|---|---|---|---|
| | overall | Placebo | | 100 mg qd | | 200 mg qd | | 50 mg bid | | 100 mg bid | |
| | p-value | (N = 79) | | (N = 68) | | (N = 75) | | (N = 73) | | (N = 79) | |
| GM-CSF | 0.0582 | 4.4 | (3.17) | −9.3 | (5.36) | −8.4 | (3.26)+ | −5.4 | (5.73) | −12.4 | (3.16)* |
| IFNγ | 0.4694 | −0.6 | (0.76) | −3.8 | (2.03) | 0.5 | (2.30) | 1.6 | (3.84) | −3.3 | (1.02) |
| IL-1βb | 0.1082 | 0.3 | (0.25) | −0.6 | (0.43) | −0.6 | (0.58) | 1.4 | (1.30) | −1.0 | (0.25) |
| IL-2 | 0.0645 | −0.3 | (0.64) | −1.9 | (0.97) | −2.0 | (0.75) | −0.2 | (0.75) | −2.3 | (0.52) |
| IL-4 | 0.8137 | −0.3 | (0.71) | 0.8 | (2.50) | 0.2 | (2.35) | −0.3 | (1.53) | −2.7 | (1.01) |
| IL-5 | 0.1622 | −0.2 | (0.46) | −0.2 | (0.25) | 0.1 | (0.55) | −18.2 | (18.08) | −0.8 | (0.31) |
| IL-6 | <0.0001 | −2.5 | (2.67) | −7.0 | (1.96)* | −9.6 | (2.34)*** | −4.2 | (2.35)* | −11.0 | (1.57)*** |
| IL-7 | 0.0011 | 0.2 | (0.56) | −0.4 | (0.35) | −2.5 | (0.88)** | −1.8 | (0.51)+ | −2.7 | (0.40)** |
| IL-8 | 0.4554 | 1.4 | (1.14) | 4.1 | (5.60) | −2.0 | (1.04) | 2.3 | (4.09) | −1.8 | (1.90) |
| IL-10 | 0.1223 | −0.6 | (1.26) | 0.1 | (0.90) | −0.5 | (1.28) | −2.1 | (1.10) | −3.3 | (0.58) |
| IL-12 | 0.0399 | 0.9 | (0.80) | −0.2 | (0.82) | −1.7 | (0.74)+ | −1.9 | (1.21)+ | −2.3 | (0.67)* |
| IL-13 | 0.1546 | 0.2 | (0.46) | −0.6 | (0.43) | −0.9 | (0.47) | −0.6 | (0.33) | −1.5 | (0.64) |
| IL-17α | 0.4753 | 0.4 | (0.77) | −5.9 | (2.64) | −1.6 | (1.75) | −4.4 | (1.91) | 3.9 | (6.20) |
| IL-21 | 0.0745 | 0.3 | (0.41) | −0.3 | (0.43) | −1.4 | (0.56) | −0.1 | (0.85) | −1.6 | (0.43)+ |
| IL-23 | 0.0486 | −75.9 | (178.16) | 38.3 | (229.09) | −435.8 | (175.50) | −772.1 | (345.88)* | −273.6 | (142.42) |
| MIP-1α | 0.2227 | −1.8 | (2.59) | 7.0 | (7.05) | −1.6 | (0.69) | 1.0 | (2.78) | −3.1 | (0.68) |
| MIP-1β | 0.0413 | 1.4 | (0.97) | −0.7 | (1.37) | −1.6 | (1.23)* | 0.4 | (1.20) | −2.1 | (0.71)* |
| TNFα | 0.3164 | 0.1 | (0.43) | 1.0 | (1.21) | 0.0 | (1.54) | −0.2 | (0.28) | −1.7 | (0.27) |

TABLE III
Week 12 observed-case table

| | Treatment | Mean (SE) change from baseline at Week 12, in pg/mL | | | | | | | | | |
|---|---|---|---|---|---|---|---|---|---|---|---|
| | overall | Placebo | | 100 mg qd | | 200 mg qd | | 50 mg bid | | 100 mg bid | |
| | p-value | (N = 79) | | (N = 68) | | (N = 75) | | (N = 73) | | (N = 79) | |
| GM- | 0.0585 | 2.9 | (4.65) | −19.5 | (12.49) | −15.0 | (4.99)+ | −14.4 | (5.00)+ | −21.1 | (4.45)* |
| CSF | | | | | | | | | | | |
| IFNγ | 0.0200 | 0.0 | (1.48) | −6.6 | (2.84)+ | −3.1 | (1.36)+ | −3.4 | (1.26)+ | −5.8 | (1.25)** |
| IL-1βb | 0.0040 | 0.2 | (0.34) | −1.2 | (0.79) | −1.6 | (0.50)** | −0.9 | (0.29) | −1.8 | (0.39)** |
| IL-2 | 0.0187 | −0.5 | (0.61) | −3.5 | (2.14) | −3.3 | (0.96)* | −1.9 | (0.67) | −4.1 | (0.84)* |
| IL-4 | 0.4816 | 1.2 | (1.68) | −1.6 | (2.13) | −2.5 | (2.30) | −2.1 | (1.16) | −2.7 | (1.49) |
| IL-5 | 0.0535 | −0.4 | (0.47) | −0.7 | (0.36) | 0.0 | (0.56) | −18.1 | (17.92) | −1.5 | (0.35) |
| IL-6 | <0.0001 | −2.6 | (2.76) | −8.0 | (2.04)** | −11.2 | (2.52)*** | −6.0 | (2.45)** | −14.1 | (2.05)*** |
| IL-7 | 0.0012 | 0.2 | (0.69) | −1.2 | (0.46) | −3.3 | (1.03)** | −2.6 | (0.63)** | −3.0 | (0.50)*** |
| IL-8 | 0.5624 | 1.0 | (1.70) | 0.7 | (4.80) | −1.6 | (2.03) | 6.9 | (5.30) | 2.4 | (4.64) |
| IL-10 | 0.1457 | −0.1 | (1.11) | −0.1 | (1.85) | −1.5 | (2.23) | −3.7 | (1.11) | −4.2 | (0.71) |
| IL-12 | 0.4203 | 0.9 | (0.92) | −1.2 | (0.88) | 20.5 | (23.04) | −2.3 | (1.28) | −3.9 | (0.70) |
| IL-13 | 0.0009 | 1.0 | (0.90) | −1.5 | (0.56)* | −2.2 | (0.54)** | −1.1 | (0.57)* | −2.9 | (0.75)*** |
| IL-17α | 0.9182 | 0.6 | (0.75) | −7.4 | (2.55) | −0.9 | (3.27) | −3.3 | (3.30) | −1.9 | (3.65) |
| IL-21 | 0.0036 | −0.1 | (0.57) | 0.5 | (0.95) | −2.3 | (0.72)+ | −1.6 | (0.66) | −2.8 | (0.56)* |
| IL-23 | 0.0384 | −248.7 | (319.00) | −337.9 | (295.71) | −949.7 | (203.69)* | −888.7 | (353.14)+ | −831.6 | (209.54)+ |
| MIP-1α | 0.1096 | −6.3 | (6.35) | 9.1 | (9.77) | −2.6 | (0.91) | 8.5 | (5.30) | −5.2 | (1.02) |
| MIP-1β | 0.3481 | 0.4 | (1.01) | −1.7 | (1.29) | −1.5 | (1.06) | −1.1 | (1.02) | −1.3 | (0.96) |
| TNFα | 0.0994 | −0.6 | (0.70) | −1.0 | (0.46) | −1.2 | (0.92) | 0.3 | (0.58) | −2.2 | (0.44) |

As shown in the table above, changes in biomarkers are seen, more particularly a reduction in IFNγ after 12 week treatment.

As shown in the table above, changes in biomarkers are seen, more particularly a reduction in GM-CSF, IL6, and IL1 after 12 week treatment.

### Example 2. Clinical Study Cutaneous Lupus

The study of the current example is a Phase 2, randomized, double-blind, placebo-controlled study to assess the safety and efficacy of a JAK inhibitor (the compound I) and a SYK inhibitor conducted in female subjects with moderately-to-severely active cutaneous lupus erythematosus (CLE). The primary objective of this study is to evaluate the efficacy of the JAK inhibitor and the SYK inhibitor in females with moderately-to-severely active cutaneous lupus erythematosus.

2.1. Study Endpoints

Primary outcome measure is:
Change from baseline in Cutaneous Lupus Erythematosus Disease Area and Severity Index (CLASI) Activity Score from baseline to week 12 [Time Frame: Baseline; Week 12] CLASI activity score measures disease activity, with higher scores indicating more severe disease.

Secondary outcome measures are:
Proportion of Participants at Week 12 with Decrease of ≥5 Points in CLASI Activity Score from Baseline [Time Frame: Baseline; Week 12]
CLASI activity score measures disease activity, with higher scores indicating more severe disease.
Proportion of Participants at Week 12 with No Worsening in CLASI Activity Score from Baseline [Time Frame: Baseline; Week 12]
CLASI activity score measures disease activity, with higher scores indicating more severe disease. Worsening was defined as ≥3 point increase in CLASI activity score.
Proportion of Participants at Week 24 with Decrease of ≥5 Points in CLASI Activity Score from Baseline [Time Frame: Baseline; Week 24]
CLASI activity score measures disease activity, with higher scores indicating more severe disease.
Proportion of Participants at Week 24 with No Worsening in CLASI Activity Score from Baseline [Time Frame: Baseline; Week 24]
CLASI activity score measures disease activity, with higher scores indicating more severe disease. Worsening was defined as ≥3 point increase in CLASI activity score.

2.2. Study Interventions

The study provides for 4 types of interventions:
Drug: compound I—200 mg tablets administered orally once daily with or without food
Drug: SYK inhibitor—30 mg tablets administered orally once daily with or without food
Drug: compound I placebo—tablets administered orally once daily with or without food
Drug: SYK inhibitor placebo—tablets administered orally once daily with or without food

2.3. Study Arms

Study participants are randomized over 4 study arms:
1. Experimental: compound I: compound I 200 mg, and, SYK inhibitor placebo for 24 weeks
   Interventions:
      Drug: compound I
      Drug: SYK inhibitor placebo
2. Experimental: SYK inhibitor: SYK inhibitor 30 mg and compound I placebo for 24 weeks
   Interventions:
      Drug: SYK inhibitor
      Drug: compound I placebo
3. Placebo Comparator: Placebo: Placebo for 12 weeks, then participants will be re-randomized to receive compound I 200 mg+SYK inhibitor placebo or SYK inhibitor 30 mg+compound I placebo through Week 24.
   Interventions:
      Drug: compound I
      Drug: SYK inhibitor
      Drug: compound I placebo
      Drug: SYK inhibitor placebo
4. Experimental: Extension Period: Participants who have not permanently discontinued study drug during the first 24 weeks may enter the subsequent 24-week extension period where they will continue to receive their assigned dose of study drug, in a blinded fashion.
   Interventions:
      Drug: compound I
      Drug: SYK inhibitor
      Drug: compound I placebo
      Drug: SYK inhibitor placebo

2.4. Eligibility Criteria

To be eligible, the subjects must meet the following criteria:
Female, ≥18 to ≤75 years of age
Must have a diagnosis of CLE, either chronic (e.g., discoid) or subacute CLE per investigator evaluation, with the following:
   Moderately-to-severely active CLE (CLASI activity score ≥10) at screening and Day 1
   Prior intolerance or inadequate response to at least one of the listed medications for the treatment of CLE
   Stable dose (defined as no change in prescription for at least 28 days prior to Day 1) of antimalarials and/or topical or oral corticosteroids is permitted during the study. Individuals who are not planning to continue these medications during the study must have discontinued them at least 28 days prior to Day 1

Key exclusion criterium is the use of prohibited concomitant medications per study protocol.

### Example 3. Clinical Study Lupus Membranous Nephropathy (LMN)

The study of the current example is a Phase 2, randomized, double-blind, multicenter study evaluating the safety and efficacy of compound I and SYK inhibitor in subjects with Lupus Membranous Nephropathy (LMN). The primary objective of this study is to evaluate the efficacy of compound I and SYK inhibitor in adults with Lupus Membranous Nephropathy (LMN).

3.1. Study Endpoints

Primary outcome measure is:
Percent Change in Urine Protein From Baseline (Day 1) to Week 16 [Time Frame: Baseline to Week 16]
Urine protein will be assessed by urinary protein excretion during a 24-hour urine collection.

Secondary outcome measures are:
Change From Baseline (Day 1) in Urine Protein at Week 16 [Time Frame: Baseline to Week 16] Urine protein will be assessed by urinary protein excretion during a 24-hour urine collection.
Change From Baseline (Day 1) in Estimated Glomerular Filtration Rate (eGFR) at Week 16 [Time Frame: Baseline to Week 16]
Change from Baseline (Day 1) in urine protein creatinine ratio (UPCR) at Week 16 [Time Frame: Baseline to Week 16] UPCR will be assessed by urine protein excretion during a 24-hour urine collection.
Proportion of Participants With Partial Remission at Week 16 [Time Frame: Week 16] Partial Remission is defined as urine protein excretion below <3 g/d and urine protein excretion decrease by ≥50% among participants with Baseline (Day 1) nephrotic range proteinuria [urine protein excretion ≥3 g/d]; or urine protein excretion decrease by ≥50% among participants with subnephrotic range proteinuria [urine protein excretion <3 g/d])
Proportion of Participants with Complete Remission [Time Frame: Week 16] Complete Remission is defined as urine protein excretion below 0.5 g/day, with no hematuria.

The study provides for 4 types of interventions:
Drug: Compound I
200 mg tablet administered orally once daily
Drug: SYK inhibitor
30 mg tablet administered orally once daily
Drug: Compound I placebo
Tablet administered orally once daily
Drug: SYK inhibitor placebo
Tablet administered orally once daily

Study participants were randomized over 3 study arms:
1. Experimental: Compound I
   Interventions:
      Drug: Compound I
      Drug: SYK inhibitor placebo
2. Experimental: SYK inhibitor
   Interventions:
      Drug: SYK inhibitor
      Drug: Compound I placebo
3. Experimental: Extended Blinded Treatment Phase
   Based on reduction in urinary protein excretion, participants will continue to receive their assigned blinded study treatment for an additional 20 weeks or continue to either study treatment per the Investigator's discretion.
   Interventions:
      Drug: Compound I
      Drug: SYK inhibitor
      Drug: Compound I placebo
      Drug: SYK inhibitor placebo

To be eligible, the subjects must meet the following criteria:
Male or female between 18 and 75 years of age, inclusive, at the time of initial informed consent
Kidney biopsy within the 18 months prior to screening with a histologic diagnosis of LMN (International Society of Nephrology [ISN] and the Renal Pathology Society [RPS] 2003 classification of lupus nephritis), either Class V alone, or Class V in combination with Class II.
Urine protein excretion ≥1.5 grams per day
Estimated glomerular filtration rate (eGFR_{MDRD})≥60 mg/min/1.73 m2 based on the MDRD formulation at screening
No evidence of active or latent TB as assessed during screening

Key exclusion criteria are:
Prior treatments as follows:
   Previous treatment with a JAK inhibitor within 3 months of Day 1
   Use of rituximab or other selective B lymphocyte depleting agents (including experimental agents) within 6 months of Day 1. Enrollment is permitted if the last dose was given >6 months and CD19-positive B cells are detectable at Screening.
Use of any concomitant prohibited medications as described in the protocol

### FINAL REMARKS

It will be appreciated by those skilled in the art that the foregoing descriptions are exemplary and explanatory in nature, and intended to illustrate the invention and its preferred embodiments. Through routine experimentation, an artisan will recognize apparent modifications and variations that may be made without departing from the spirit of the invention. All such modifications coming within the scope of the appended claims are intended to be included therein. Thus, the invention is intended to be defined not by the above description, but by the following claims and their equivalents.

All publications, including but not limited to patents and patent applications, cited in this specification are herein incorporated by reference as if each individual publication are specifically and individually indicated to be incorporated by reference herein as though fully set forth.

It should be understood that factors such as the differential cell penetration capacity of the various compounds can contribute to discrepancies between the activity of the compounds in the in vitro biochemical and cellular assays.

At least some of the chemical names of compounds of the invention as given and set forth in this application, may have been generated on an automated basis by use of a commercially available chemical naming software program, and have not been independently verified. Representative programs performing this function include the Lexichem naming tool sold by Open Eye Software, Inc. and the Autonom Software tool sold by MDL, Inc. In the instance where the indicated chemical name and the depicted structure differ, the depicted structure will control.

### REFERENCES

Aletaha, D., Neogi, T., Silman, A. J., Funovits, J., Felson, D. T., Bingham, C. O., Birnbaum, N. S., Burmester, G. R., Bykerk, V. P., Cohen, M. D., Combe, B., Costenbader, K. H., Dougados, M., Emery, P., Ferraccioli, G., Hazes, J. M. W., Hobbs, K., Huizinga, T. W. J., Kavanaugh, A., Kay, J., Kvien, T. K., Laing, T., Mease, P., Ménard, H. A., Moreland, L. W., Naden, R. L., Pincus, T., Smolen, J. S., Stanislawska-Biernat, E., Symmons, D., Tak, P. P., Upchurch, K. S., Vencovsk, J., Wolfe, F., Hawker, G., 2010. 2010 Rheumatoid arthritis classification criteria: An American College of Rheumatology/European League Against Rheumatism collaborative initiative. Arthritis Rheum. 62, 2569-2581. doi:10.1002/art.27584
Alkhalifah, A, Alsantali, A., Wang, E., McElwee, K. J., Shapiro, J., 2010. Alopecia areata update: Part I. Clinical picture, histopathology, and pathogenesis. J. Am. Acad. Dermatol. 62, 177-188. doi:10.1016/j jaad.2009.10.032
Allam, M., Riad, H., 2013. Concise review of recent studies in vitiligo. Qatar Med. J. 2013, 1-19. doi:10.5339/qmj 0.2013.10
Baughman, R. P., Lower, E. E., du Bois, R. M., 2003. Sarcoidosis. The Lancet 361, 1111-1118. doi: 10.1016/S0140-6736(03)12888-7
Di Lernia, V., Bardazzi, F., 2016. Profile of tofacitinib citrate and its potential in the treatment of moderate-to-severe chronic plaque psoriasis. Drug Des. Devel. Ther. 10, 533-539. doi:10.2147/DDDT. 582599
Ezzedine, K., Lim, H. W., Suzuki, T., Katayama, I., Hamzavi, I., Lan, C. C. E., Goh, B. K., Anbar, T., de Castro, C. S., Lee, A. Y., Parsad, D., van Geel, N., Le Poole, I. C., Oiso, N., Benzekri, L., Spritz, R., Gauthier, Y., Hann, S K, Picardo, M., Taieb, A., 2012. Revised classification/nomenclature of vitiligo and related issues: the Vitiligo Global Issues Consensus Conference. Pigment Cell Melanoma Res. 25, E1-13. doi:10.1111/j.1755-148X.2012.00997.x
Gigante, A., Gasperini, M. L., Afeltra, A., Barbano, B., Margiotta, D., Cianci, R., De Francesco, I., Amoroso, A., 2011. Cytokines expression in SLE nephritis. Eur. Rev. Med. Pharmacol. Sci. 15, 15-24.
Hamzavi I, Jain H, McLean D, Shapiro J, Zeng H, Lui H, 2004. Parametric modeling of narrowband uv-b phototherapy for vitiligo using a novel quantitative tool: The vitiligo area scoring index. Arch. Dermatol. 140, 677-683. doi:10.1001/archderm.140.6.677
Iannuzzi, M. C., Rybicki, B. A., Teirstein, A. S., 2007. Sarcoidosis. N. Engl. J. Med. 357, 2153-2165. doi:10.1056/NEJMra071714
Karia, S. B., De Sousa, A., Shah, N., Sonavane, S., Bharati, A., 2015. Psychiatric morbidity and quality of life in skin diseases: A comparison of alopecia areata and psoriasis. Ind. Psychiatry J. 24, 125-128. doi:10.4103/0972-6748.181724
Klaeschen, A. S., Wenzel, J., 2016. Upcoming therapeutic targets in cutaneous lupus erythematous. Expert Rev. Clin. Pharmacol. 9, 567-578. doi:10.1586/17512433.2016.1145543
Klein, R. S., Morganroth, P. A., Werth, V. P., 2010. Cutaneous Lupus and the CLASI Instrument. Rheum. Dis. Clin. North Am. 36, 33-51. doi:10.1016/j.rdc.2009.12.001
Kolasinski, S. L., Chung, J. B., Albert, D. A., 2002. What do we know about lupus membranous nephropathy? An analytic review. Arthritis Care Res. 47, 450-455. doi:10.1002/art.10417
Kuhn, A., Krammer, P. H., Kolb-Bachofen, V., 2006. Pathophysiology of cutaneous lupus erythematosus—novel aspects. Rheumatology 45, iii14-iii16. doi:10.1093/rheumatology/ke1284
Lewis, E. J., Schwartz, M. M., Korbet, S. M., Chan, D. T. M. (Eds.), 2010. Lupus Nephritis, Second Edition. ed, Oxford Clinical Nephrology Series. Oxford University Press, Oxford, New York.
Menet, C. J. M., Smits, K. K., 2010. 5-Phenyl-[1,2,4]triazolo[1,5-A]pyridin-2-Yl Carboxamides as Jak Inhibitors. WO2010149769 (A1).
Olsen, E. A., Hordinsky, M. K., Price, V. H., Roberts, J. L., Shapiro, J., Canfield, D., Duvic, M., King Jr., L. E., McMichael, A. J., Randall, V. A., Turner, M. L., Sperling, L., Whiting, D. A., Norris, D., 2004. Alopecia areata investigational assessment guidelines-Part II. J. Am. Acad. Dermatol. 51, 440-447. doi:10.1016/j.jaad.2003.09.032
O'Shea, J. J., Laurence, A., McInnes, I. B., 2013. Back to the Future: Oral targeted therapy for RA and other autoimmune diseases. Nat. Rev. Rheumatol. 9, 173-182. doi:10.1038/nrrheum.2013.7
O'Shea, J. J., Plenge, R., 2012. JAKs and STATs in Immunoregulation and Immune-Mediated Disease. Immunity 36, 542-550. doi:10.1016/j.immuni.2012.03.014
Ponte, C., Rodrigues, A. F., O'Neill, L., Luqmani, R. A., 2015. Giant cell arteritis: Current treatment and management. World J. Clin. Cases WJCC 3, 484-494. doi:10.12998/wjcc.v3.i6.484
Proven, A., Gabriel, S. E., Orces, C., O'Fallon, W. M., Hunder, G. G., 2003. Glucocorticoid therapy in giant cell arteritis: duration and adverse outcomes. Arthritis Rheum. 49, 703-708. doi:10.1002/art.11388
Rashighi, M., Agarwal, P., Richmond, J. M., Harris, T. H., Dresser, K., Su, M., Zhou, Y., Deng, A., Hunter, C. A., Luster, A. D., Harris, J. E., 2014. CXCL10 is critical for the progression and maintenance of depigmentation in a mouse model of vitiligo. Sci. Transl. Med. 6, 223ra23. doi:10.1126/scitranslmed.3007811
Remedy for Sarcoidosis and Method of Treating the Same, 2006. JPWO2005002623 (A1) Abstract of corresponding document: EP1642592 (A1).
Rizzato, G., Riboldi, A., Imbimbo, B., Torresin, A., Milani, S., 1997. The long-term efficacy and safety of two different corticosteroids in chronic sarcoïdosis. Respir. Med. 91, 449-460. doi: 10.1016/S 0954-6111(97)90109-8
Robinson, E. S., Werth, V. P., 2015. The role of cytokines in the pathogenesis of cutaneous lupus erythematosus. Cytokine, Skin Disease, Immune Response and Cytokines 73, 326-334. doi:10.1016/j.cyto.2015.01.031
Rosenbaum, J. T., Pasadhika, S., Crouser, E. D., Choi, D., Harrington, C. A., Lewis, J. A., Austin, C. R., Diebel, T. N., Vance, E. E., Braziel, R. M., Smith, J. R., Planck, S. R., 2009. Hypothesis: Sarcoidosis is a STAT1-mediated disease. Clin. Immunol. Orlando Fla. 132, 174-183. doi:10.1016/j.clim.2009.04.010
Rutgers, A., Mulder, D. J., Brouwer, E., 2016. Giant cell arteritis, truly a form of systemic vasculitis. Neth. J. Med. 74, 180-181.
Shepherd, J., Nicklin, M. J. H., 2005. Elastic-Vessel Arteritis in Interleukin-1 Receptor Antagonist-Deficient Mice Involves Effector Th1 Cells and Requires Interleukin-1 Receptor. Circulation 111, 3135-3140. doi:10.1161/CIRCULATIONAHA.104.519132
Singh, S., Wu, T., Xie, C., Vanarsa, K., Han, J., Mahajan, T., Oei, H. B., Ahn, C., Zhou, X. J., Putterman, C., Saxena, R., Mohan, C., 2012. Urine VCAM-1 as a marker of renal pathology activity index in lupus nephritis. Arthritis Res. Ther. 14, R164. doi:10.1186/ar3912
The Merck Manual of Diagnosis and Therapy, 19th ed, 2011. MERCK SHARP & DOHME CORP., A SUBSIDIARY OF MERCK & CO., INC., Whitehouse Station, NJ, USA.
Vainchenker, W., Dusa, A., Constantinescu, S. N., 2008. JAKs in pathology: Role of Janus kinases in hematopoietic malignancies and immunodeficiencies. Semin. Cell Dev. Biol. 19, 385-393. doi: 10.1016/jsemcdb0.2008.07.002
Van't Klooster, G., Brys, R. C. X., Van, R., Namour, F. S., 2013. Aminotriazolopyridine for Use in the Treatment of Inflammation, and Pharmaceutical Compositions Thereof. WO2013189771 (A1).
Wang, E., McElwee, K. J., 2011. Etiopathogenesis of alopecia areata: Why do our patients get it? Dermatol. Ther. 24, 337-347. doi:10.1111/j.1529-8019.2011.01416.x
Wang, S., Yang, N., Zhang, L., Huang, B., Tan, H., Liang, Y., Li, Y., Yu, X., 2010. Jak/STAT signaling is involved in the inflammatory infiltration of the kidneys in MRL/lpr mice. Lupus 19, 1171-1180. doi:10.1177/0961203310367660
Wigerinck, P., Van't Klooster, G. A. E., 2016. Methods for the Treatment of Cardiovascular Disorders. WO2016165952 (A1).
Wigerinck, P., Van't Klooster, G. A. E., Vanhoutte, F., 2016. Methods for the Treatment of Inflammatory Disorders. WO2016165953 (A1).
Xing, L., Dai, Z., Jabbari, A., Cerise, J. E., Higgins, C. A., Gong, W., de Jong, A., Harel, S., DeStefano, G. M., Rothman, L., Singh, P., Petukhova, L., Mackay-Wiggan, J., Christiano, A. M., Clynes, R., 2014. Alopecia areata is driven by cytotoxic T lymphocytes and is reversed by JAK inhibition. Nat. Med. 20, 1043-1049. doi:10.1038/nm.3645
Yamaoka, K., 2016. Janus kinase inhibitors for rheumatoid arthritis. Curr. Opin. Chem. Biol. 32, 29-33. doi:10.1016/j.cbpa.2016.03.006

1. A compound according to Formula I: 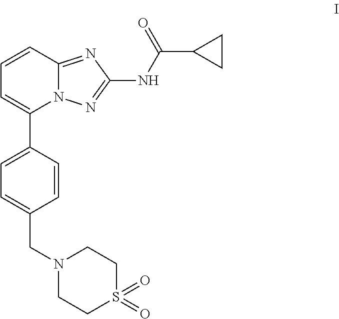 [SMILES: O=C(Nc1nc2cccc(-c3ccc(CN4CCS(=O)(=O)CC4)cc3)n2n1)C1CC1]
or a pharmaceutically acceptable salt thereof, or a solvate or the salt of a solvate thereof, or an active metabolite thereof for use in the prophylaxis and/or treatment of alopecia areata, vitiligo, cutaneous lupus, lupus nephritis, giant cell arteritis, sarcoïdosis, and/or a sarcoïdosis-related condition.

2. The compound for use according to claim 1, wherein the active metabolite is according to Formula II:

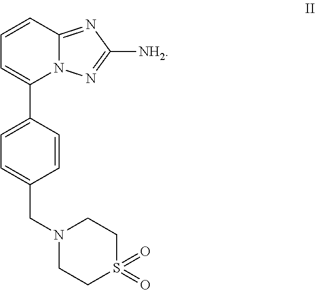 [SMILES: Nc1nc2cccc(-c3ccc(CN4CCS(=O)(=O)CC4)cc3)n2n1]

3. A compound for use according to claim 1, wherein the compound according to Formula I is a pharmaceutically acceptable salt, wherein said salt is formed with a salt forming agent selected from a hydrobromic acid, hydrochloric acid, sulfuric acid, toluenesulfonic acid, benzenesulfonic acid, oxalic acid, maleic acid, naphthalene-2-sulfonic acid, naphthalene-1,5-disulfonic acid, 1-2-ethane disulfonic acid, methanesulfonic acid, 2-hydroxy ethanesulfonic acid, phosphoric acid, ethane sulfonic acid, malonic acid, 2-5-dihydroxybenzoic acid, or L-Tartaric acid.

4. A compound for use according to claim 1, wherein the compound according to Formula I is a pharmaceutically acceptable salt, wherein said salt is formed with maleic acid.

5. A compound, or a pharmaceutically acceptable salt thereof for use according to any one of claims 1-4, in combination with a further therapeutic agent.

6. A pharmaceutical composition for use in the prophylaxis and/or treatment of alopecia areata, vitiligo, cutaneous lupus, lupus nephritis, giant cell arteritis, sarcoïdosis, and/or a sarcoïdosis-related condition comprising the compound, or a pharmaceutically acceptable salt thereof according to any one of claims 1-4, and a pharmaceutically acceptable carrier, excipient, or diluent.

7. A pharmaceutical composition for use according to claim 6, comprising a further therapeutic agent.

8. The compound or a pharmaceutically acceptable salt thereof, according to claim 5 or the pharmaceutical composition for use according to claim 7, wherein the further therapeutic agent is an agent for the prophylaxis and/or treatment of alopecia areata.

9. A compound, or a pharmaceutically acceptable salt thereof, for use according to any one of claims 1-4, or a pharmaceutical composition according to claim 6 or 7 in an individual afflicted with alopecia areata, vitiligo, cutaneous lupus, lupus nephritis, giant cell arteritis, sarcoïdosis, and/or a sarcoïdosis-related condition.

10. A compound, or a pharmaceutically acceptable salt thereof, or a pharmaceutical composition for use according to any one of claims 1-9, wherein the compound is administered at a dose of 100 mg b.i.d.

11. A compound, or a pharmaceutically acceptable salt thereof, or a pharmaceutical composition for use according to any one of claims 1-9, wherein the compound is administered at a dose of 200 mg qd.

12. A compound, or a pharmaceutically acceptable salt thereof, or a pharmaceutical composition for use according to any one of claims 1-11, wherein the compound is administered over a period of least 4, 6, 8, 10, 12, 14, 16, 20, or 24 weeks.

13. A compound, or a pharmaceutically acceptable salt thereof, or a pharmaceutical composition for use according to any one of claims 1-11, wherein the compound is administered over a period of least 12 weeks.

14. A compound, or a pharmaceutically acceptable salt thereof, for use according to any one of claims 1-4, or a pharmaceutical composition according to claim 6 or 7 in an individual afflicted with alopecia areata, as characterized by the Severity Alopecia Tool (SALT) score.

15. A compound, or a pharmaceutically acceptable salt thereof, for use according to any one of claims 1-4, or a pharmaceutical composition according to claim 6 or 7 in an individual afflicted with alopecia areata, as characterized by a SALT score of at least 25%.

16. A compound, or a pharmaceutically acceptable salt thereof, for use according to any one of claims 1-4, or a pharmaceutical composition according to claim 6 or 7 in an individual afflicted with alopecia areata, as characterized by a SALT score of at least 50%.

17. A compound, or a pharmaceutically acceptable salt thereof, for use according to any one of claims 1-4, or a pharmaceutical composition according to claim 6 or 7 in an individual afflicted with alopecia areata, as characterized by a SALT score of at least 75%.

18. A compound, or a pharmaceutically acceptable salt thereof, for use according to any one of claims 1-4, or a pharmaceutical composition according to claim 6 or 7, wherein the regrowth improvement is at least 10% (SALT₁₀), 20% (SALT₂₀), 30% (SALT₃₀), 40% (SALT₄₀), or 50% (SALT₅₀) after treatment compared to before treatment.

19. A method for the treatment the prophylaxis and/or treatment of alopecia areata comprising the steps of:
a) measuring the SALT score of an individual by performing a scalp hair loss analysis by assessing the hair loss on the left side, right side, top and back of the patient, wherein the left side and the right side each account for 18% of the overall head surface, the top accounts for 40% of the overall head surface and the back accounts for 24% of the overall head surface,
b) assessing the type of hair remaining on each area (pigmented/non-pigmented/indeterminant),
c) calculating a SALT score by the formula:
d) SALT score=(% left side)*0.18+(% right side)*0.18+(% top side)*0.40+(% back side)*0.24, and
e) determining a daily dose of the compound according to Formula I, or a pharmaceutically acceptable salt thereof comprised between 25 mg and 400 mg for administration to said individual.

20. A compound, or a pharmaceutically acceptable salt thereof, for use according to any one of claims 1-4, or a pharmaceutical composition according to claim 6 or 7 in an individual afflicted with alopecia areata, said use comprising the steps of
a) assaying a blood sample,
b) measuring levels of at least one alopecia areata-associated biomarker,
c) administering a therapeutically effective amount of the compound, or a pharmaceutically acceptable salt thereof to the individual,
d) measuring the post-treatment level of said alopecia areata-associated biomarker and comparing with pre-treatment levels, and
e) adjusting the dosage of compound, or a pharmaceutically acceptable salt thereof to be administered to the individual, wherein said dosage is increased when a decrease of less than 2% after 12 weeks in levels of said alopecia areata-associated biomarker is detected.

21. A compound, or a pharmaceutically acceptable salt thereof, for use according to claim 20, wherein the biomarker is IFN_{γ}.

22. A compound, or a pharmaceutically acceptable salt thereof, for use according to claim 20, wherein the biomarker level is decreased by at least 5%, at least 10%, or at least 15% compared to pre-treatment levels.

23. A compound, or a pharmaceutically acceptable salt thereof, or a pharmaceutical composition for use according to claim 20, wherein the IFN_{γ} are decreased by at least 5% compared to pre-treatment levels after 12 weeks treatment.

24. The compound or a pharmaceutically acceptable salt thereof, according to claim 5 or the pharmaceutical composition for use according to claim 7, wherein the further therapeutic agent is an agent for the prophylaxis and/or treatment of vitiligo.

25. A compound, or a pharmaceutically acceptable salt thereof, for use according to any one of claims 1-5, or a pharmaceutical composition according to claim 6 or 7 in an individual afflicted with vitiligo.

26. A compound, or a pharmaceutically acceptable salt thereof, or a pharmaceutical composition for use according to any one of claim 1-7, 24, or 25, wherein the compound is administered at a dose of 100 mg b.i.d.

27. A compound, or a pharmaceutically acceptable salt thereof, or a pharmaceutical composition for use according to any one of claim 1-7, 24, or 25, wherein the compound is administered at a dose of 200 mg qd.

28. A compound, or a pharmaceutically acceptable salt thereof, or a pharmaceutical composition for use according to any one of claim 1-9, 24, or 25, wherein the compound is administered over a period of least 4, 6, 8, 10, 12, 14, 16, 20, or 24 weeks.

29. A compound, or a pharmaceutically acceptable salt thereof, or a pharmaceutical composition for use according to any one of claim 1-9, 24, or 25, wherein the compound is administered over a period of least 12 weeks.

30. A compound, or a pharmaceutically acceptable salt thereof, for use according to any one of claims 1-5, or a pharmaceutical composition according to claim 24 or 25 in an individual afflicted with vitiligo, as characterized by the Vitiligo Activity Severity Index (VAST) score.

31. A compound, or a pharmaceutically acceptable salt thereof, for use according to any one of claims 1-5, or a pharmaceutical composition according to claim 24 or 25 in an individual afflicted with vitiligo, as characterized by a VASI score of at least 25%.

32. A compound, or a pharmaceutically acceptable salt thereof, for use according to any one of claims 1-5, or a pharmaceutical composition according to claim 24 or 25 in an individual afflicted with vitiligo, as characterized by a VASI score of at least 50%.

33. A compound, or a pharmaceutically acceptable salt thereof, for use according to any one of claims 1-5, or a pharmaceutical composition according to claim 24 or 25 in an individual afflicted with vitiligo, as characterized by a VASI score of at least 75%.

34. A compound, or a pharmaceutically acceptable salt thereof, for use according to any one of claims 1-5, or a pharmaceutical composition according to claim 24 or 25, wherein the VASI improvement is at least 10%, 20%, 30%, 40%, or 50% after treatment compared to before treatment.

35. A method for the treatment the prophylaxis and/or treatment of vitiligo comprising the steps of:
a) measuring the depigmentation of the hands, upper extremities (excluding hands), trunk, lower extremities (excluding the feet), and feet of an individual using the hand as a unit (palm plus the volar surface of all the digits),
b) calculating a VASI score by the formula:
VASI=Σ_{all body site}[Hand Unit]×[Residual Depigmentation] a.
c) determining a daily dose of the compound according to Formula I, or a pharmaceutically acceptable salt thereof comprised between 25 mg and 400 mg for administration to said individual.

36. A compound, or a pharmaceutically acceptable salt thereof, for use according to any one of claim 1-5, or a pharmaceutical composition according to claim 24 or 25 in an individual afflicted with vitiligo, said use comprising the steps of:
a) assaying a blood sample,
b) measuring levels of at least one vitiligo-associated biomarker,
c) administering a therapeutically effective amount of the compound, or a pharmaceutically acceptable salt thereof to the individual,
d) measuring the post-treatment level of said vitiligo-associated biomarker and comparing with pre-treatment levels, and
e) adjusting the dosage of the compound, or a pharmaceutically acceptable salt thereof to be administered to the individual, wherein said dosage is increased when a decrease of less than 2% after 12 weeks in levels of said vitiligo-associated biomarker is detected.

37. A compound, or a pharmaceutically acceptable salt thereof, for use according to claim 36, wherein the biomarker is IFN_{γ}.

38. A compound, or a pharmaceutically acceptable salt thereof, for use according to claim 36, wherein the biomarker level is decreased by at least 5%, at least 10%, or at least 15% compared to pre-treatment levels.

39. A compound, or a pharmaceutically acceptable salt thereof, or a pharmaceutical composition for use according to claim 36, wherein the IFN_{γ} are decreased by at least 5% compared to pre-treatment levels after 12 weeks of treatment.

40. A pharmaceutical composition for use in the prophylaxis and/or treatment of cutaneous lupus, comprising the compound according to Formula I, or a pharmaceutically acceptable salt thereof, and a pharmaceutically acceptable carrier, excipient, or diluent.

41. A pharmaceutical composition for use according to claim 40, comprising a further therapeutic agent.

42. A compound, or a pharmaceutically acceptable salt thereof, for use according to any one of claims 1-5, or a pharmaceutical composition for use according to claim 40 or 41 in an individual afflicted with cutaneous lupus.

43. A method of treating cutaneous lupus in a human, which comprises administering to an individual afflicted with cutaneous lupus, a therapeutically effective amount of compound, according to any one of claims 1-4, or a pharmaceutically acceptable salt or solvate thereof.

44. The method according to claim 43, wherein said method comprises administering at least one further therapeutic agent.

45. The compound, or a pharmaceutically acceptable salt thereof, for use according to claim 5, or the pharmaceutical composition for use according to claim 41, or the method according to claim 44, wherein the further therapeutic agent is selected from angiotensin converting enzyme (ACE) inhibitors, angiotensin receptor blockers (ARBs), antimalarials, statins, cyclophosphamide, azathioprine, 6-mercaptopurine, abatacept, rituximab, belimumab, cyclosporine and other calcineurin inhibitors.

46. A compound, or a pharmaceutically acceptable salt thereof, for use according to any one of claims 1-5, or a pharmaceutical composition according for use according to claim 40 or 42, or the method according to claim 43, wherein said cutaneous lupus is selected from the group consisting of chronic cutaneous lupus erythematosus, subacute cutaneous lupus erythematosus, discoid lupus erythematosus, acute cutaneous lupus erythematosus, and drug-induced lupus erythematosus.

47. A compound, or a pharmaceutically acceptable salt thereof, for use according to any one of claims 1-5, or a pharmaceutical composition according for use according to claim 40 or 41, wherein the administration of said compound, or a pharmaceutically acceptable salt thereof, or a pharmaceutical composition results in detectable changes of a biomarker.

48. A compound, or a pharmaceutically acceptable salt thereof, for use according to any one of claims 1-5, or a pharmaceutical composition according to claim 40 or 41 in an individual afflicted with cutaneous lupus, said use comprising the steps of:
a) assaying a blood sample,
b) measuring levels of at least one cutaneous lupus-associated biomarker,
c) administering a therapeutically effective amount of the compound, or a pharmaceutically acceptable salt thereof to the individual,
d) measuring the post-treatment level of said cutaneous lupus-associated biomarker and comparing with pre-treatment levels, and
e) adjusting the dosage of the compound, or a pharmaceutically acceptable salt thereof to be administered to the individual, wherein said dosage is increased when a decrease of less than 2% after 12 weeks in levels of said cutaneous lupus-associated biomarker is detected.

49. A compound, or a pharmaceutically acceptable salt thereof, or a pharmaceutical composition for use according to claim 47 or 48, wherein the biomarker is selected from the group consisting of IL-6 and a complement component.

50. A compound, or a pharmaceutically acceptable salt thereof, or a pharmaceutical composition for use according to claim 47 or 48, wherein the biomarker is IL6.

51. A compound, or a pharmaceutically acceptable salt thereof, or a pharmaceutical composition for use according to claim 47 or 48, wherein the biomarker level is decreased by at least 5%, at least 10%, at least 15%, at least 20%, at least 30%, at least 40%, at least 50%, at least 60%, at least 70%, at least 80%, at least 90% compared to pre-treatment levels.

52. A compound, or a pharmaceutically acceptable salt thereof, for use according to any one of claims 1-5, or a pharmaceutical composition according for use according to claim 40 or 41, or the method according to claim 43 or 44, in an individual afflicted with cutaneous lupus as characterized by the Cutaneous Lupus Erythematosus Disease Area and Severity Index (CLASI).

53. A compound, or a pharmaceutically acceptable salt thereof, for use according to any one of claims 1-5, or a pharmaceutical composition according for use according to claim 40 or 41, or the method according to claim 43 or 44, wherein the compound is administered at a dose of 100 mg b.i.d.

54. A compound, or a pharmaceutically acceptable salt thereof, for use according to any one of claims 1-5, or a pharmaceutical composition according for use according to claim 40 or 41, or the method according to claim 43 or 44, wherein the compound is administered at a dose of 200 mg qd.

55. A compound, or a pharmaceutically acceptable salt thereof, for use according to any one of claims 1-5, or a pharmaceutical composition according for use according to claim 40 or 41, or the method according to claim 43 or 44, wherein the compound is administered over a period of at least 6 months.

56. A pharmaceutical composition for use in the prophylaxis and/or treatment of lupus nephritis, comprising the compound according to Formula I, or a pharmaceutically acceptable salt thereof, and a pharmaceutically acceptable carrier, excipient, or diluent.

57. A pharmaceutical composition for use according to claim 56, comprising a further therapeutic agent.

58. A compound, or a pharmaceutically acceptable salt thereof, for use according to any one of claims 1-5, or a pharmaceutical composition for use according to claim 56 or 57 in an individual afflicted with lupus nephritis.

59. A method of treating lupus nephritis in a human, which comprises administering to a patient having lupus nephritis a therapeutically effective amount of compound, according to any one of claims 1-6, or a pharmaceutically acceptable salt or solvate thereof.

60. A compound, or a pharmaceutically acceptable salt thereof, for use according to any one of claims 1-5, or a pharmaceutical composition according for use according to claim 56 or 57, or the method according to claim 59, wherein said lupus nephritis is membranous lupus nephritis.

61. The method according to claim 59, wherein said method comprises administering a further therapeutic agent.

62. The compound, or a pharmaceutically acceptable salt thereof for use according to claim 5, or a pharmaceutical composition for use according to claim 57, or the method according to claim 61, wherein further therapeutic agent is selected from angiotensin converting enzyme (ACE) inhibitors, angiotensin receptor blockers (ARBs), antimalarials, statins, cyclophosphamide, azathioprine, 6-mercaptopurine, abatacept, rituximab, belimumab, cyclosporine and other calcineurin inhibitors.

63. A compound, or a pharmaceutically acceptable salt thereof, for use according to any one of claims 1-5, or a pharmaceutical composition according for use according to claim 56 or 57, wherein the administration of said compound, or a pharmaceutically acceptable salt thereof, or a pharmaceutical composition results in detectable changes of a biomarker.

64. A method of treating lupus nephritis in an individual afflicted with lupus nephritis, comprising the steps of:
a) assaying a blood sample,
b) measuring levels of at least one lupus nephritis-associated biomarker,
c) administering a therapeutically effective amount of the compound, or a pharmaceutically acceptable salt thereof to the individual,
d) measuring the post-treatment level of said lupus nephritis-associated biomarker and comparing with pre-treatment levels, and
e) adjusting the dosage of the compound, or a pharmaceutically acceptable salt thereof to be administered to the individual, wherein said dosage is increased when a decrease of less than 2% after 12 weeks in levels of said lupus nephritis-associated biomarker is detected.

65. A compound, or a pharmaceutically acceptable salt thereof, or a pharmaceutical composition for use according to claim 63, or the method according to claim 64, wherein the biomarker is selected from the group consisting of IL-6, IL-10, IFNγ and TGFβ.

66. A compound, or a pharmaceutically acceptable salt thereof, or a pharmaceutical composition for use according to claim 63, or the method according to claim 64, wherein the biomarker is IL6.

67. A compound, or a pharmaceutically acceptable salt thereof, or a pharmaceutical composition for use according to claim 63, or the method according to claim 64, wherein the biomarker level is decreased by at least 5%, at least 10%, at least 15%, at least 20%, at least 30%, at least 40%, at least 50%, at least 60%, at least 70%, at least 80%, at least 90% compared to pre-treatment level.

68. A compound, or a pharmaceutically acceptable salt thereof, for use according to any one of claims 1-5, or a pharmaceutical composition according for use according to claim 56 or 57, or the method of any claim 59 or 61, wherein the administration of said compound, or a pharmaceutically acceptable salt thereof, or said pharmaceutical composition reduces proteinuria in the subject.

69. A compound, or a pharmaceutically acceptable salt thereof, or a pharmaceutical composition or the method of claim 68, wherein proteinuria reduction is measured by 24 hour urine protein, 24 hour protein to creatinine ratio, spot protein to creatinine ratio, 24 hour urine albumin, 24 hour albumin to creatinine ratio, spot albumin to creatinine ratio, or by a urinary dipstick.

70. A compound, or a pharmaceutically acceptable salt thereof, or a pharmaceutical composition or the method of claim 68, wherein the periodic administration of said compound, or a pharmaceutically acceptable salt thereof reduces the subject's protein to creatinine ratio.

71. A compound, or a pharmaceutically acceptable salt thereof, or a pharmaceutical composition or the method of claim 70, wherein the subject's protein to creatinine ratio is reduced by at least 50% as compared to baseline.

72. A compound, or a pharmaceutically acceptable salt thereof, for use according to any one of claims 1-5, or a pharmaceutical composition according for use according to claim 56 or 57, or the method of any claim 59 or 61, wherein the compound is administered at a dose of 100 mg b.i.d.

73. A compound, or a pharmaceutically acceptable salt thereof, for use according to any one of claims 1-5, or a pharmaceutical composition according for use according to claim 56 or 57, or the method of any claim 59 or 61, wherein the compound is administered at a dose of 200 mg qd.

74. A compound, or a pharmaceutically acceptable salt thereof, or a pharmaceutical composition or the method according to claim 72 or 73, wherein the compound is administered over a period of at least 6 months.

75. A pharmaceutical composition for use in the prophylaxis and/or treatment of sarcoïdosis, comprising the compound according to Formula I, or a pharmaceutically acceptable salt thereof, and a pharmaceutically acceptable carrier, excipient, or diluent.

76. A pharmaceutical composition for use according to claim 75, comprising a further therapeutic agent.

77. A compound, or a pharmaceutically acceptable salt thereof, for use according to any one of claims 1-5, or a pharmaceutical composition for use according to claim 75 or 76 in an individual afflicted with sarcoïdosis.

78. A method of treating sarcoïdosis in a human, which comprises administering to a patient having sarcoïdosis a therapeutically effective amount of compound, according to any one of claims 1-5, or a pharmaceutically acceptable salt or solvate thereof.

79. The method according to claim 78, further comprising the administration of a further therapeutic agent.

80. The method according to claim 79, wherein the further therapeutic agent is selected from anti-inflammatory agent, a steroid, an immunosuppressant compound, and an antibiotic

81. The method according to claim 79, wherein the further therapeutic agent is selected from corticosteroid, prednisone, methotrexate, azathioprine, hydroxychloroquine, cyclophosphamide, minocycline, doxycycline, chloroquin, infliximab, a penicillin antibiotic, a cephalosporin antibiotic, a macrolide antibiotic, a lincomycin antibiotic, and a tetracycline antibiotic.

82. The compound, or a pharmaceutically acceptable salt thereof, a pharmaceutical composition or the method according to any one of claim 1-5, or 75-81, wherein said sarcoïdosis is selected from the group consisting of cardiac sarcoïdosis, cutaneous sarcoïdosis, hepatic sarcoïdosis, pulmonary sarcoïdosis, neurosarcoïdosis, Lofgren's syndrome, and chronic cutaneous sarcoïdosis.

83. The compound, or a pharmaceutically acceptable salt thereof, a pharmaceutical composition or the method according to any one of claim 1-5, or 75-81, wherein said sarcoïdosis is cutaneous sarcoïdosis.

84. The compound, or a pharmaceutically acceptable salt thereof, a pharmaceutical composition or the method according to any one of claim 1-5, or 75-81, wherein said sarcoïdosis is Löfgren's syndrome

85. The compound, or a pharmaceutically acceptable salt thereof, a pharmaceutical composition or the method according to any one of claim 1-5, or 75-81, wherein said sarcoïdosis is neurosarcoïdosis

86. The compound, or a pharmaceutically acceptable salt thereof, a pharmaceutical composition or the method according to any one of claim 1-5, or 75-81, wherein said sarcoïdosis is cardiac sarcoïdosis

87. The compound, or a pharmaceutically acceptable salt thereof, a pharmaceutical composition or the method according to any one of claim 1-5, or 75-81, wherein said sarcoïdosis is pulmonary sarcoïdosis.

88. The compound, or a pharmaceutically acceptable salt thereof, a pharmaceutical composition or the method according to any one of claim 1-5, or 75-81, wherein said sarcoïdosis is pulmonary fibrosis caused by pulmonary sarcoïdosis.

89. A compound, or a pharmaceutically acceptable salt thereof, for use according to any one of claims 1-5, or a pharmaceutical composition according for use according to claim 75 or 77, or the method according to claim 78 or 79 wherein the administration of said compound, or a pharmaceutically acceptable salt thereof, or a pharmaceutical composition results in detectable changes of a biomarker.

90. A method of treating sarcoïdosis in an individual afflicted with sarcoïdosis, comprising the steps of:
a) assaying a blood sample,
b) measuring levels of at least one sarcoïdosis and/or a sarcoïdosis-related condition-associated biomarker,
c) administering a therapeutically effective amount of the compound, or a pharmaceutically acceptable salt thereof to the individual,
d) measuring the post-treatment level of said sarcoïdosis and/or a sarcoïdosis-related condition-associated biomarker and comparing with pre-treatment levels, and
e) adjusting the dosage of compound, or a pharmaceutically acceptable salt thereof to be administered to the individual, wherein said dosage is increased when a decrease of less than 2% after 12 weeks in levels of said sarcoïdosis and/or a sarcoïdosis-related condition-associated biomarker is detected.

91. The compound, or a pharmaceutically acceptable salt thereof, or a pharmaceutical composition according for use according to claim 89 or the method according to claim 90, wherein the biomarker is CXCL9 or CXCL10.

92. The compound, or a pharmaceutically acceptable salt thereof, or a pharmaceutical composition according for use according to claim 89 or the method according to claim 90, wherein the biomarker is CXCL10.

93. The compound, or a pharmaceutically acceptable salt thereof, or a pharmaceutical composition according for use according to claim 89 or the method according to claim 90, wherein the biomarker level is decreased by at least 5%, at least 10%, at least 15%, at least 20%, at least 30%, at least 40%, at least 50%, at least 60%, at least 70%, at least 80%, at least 90% compared to pre-treatment levels.

94. A compound, or a pharmaceutically acceptable salt thereof, for use according to any one of claims 1-5, or a pharmaceutical composition according for use according to claim 75 or 77, or the method according to claim 78 or 79, wherein the administration of said compound, or a pharmaceutically acceptable salt thereof, or said pharmaceutical composition results in a detectable improvement in one or more symptoms of said sarcoïdosis.

95. A compound, or a pharmaceutically acceptable salt thereof, or the pharmaceutical composition or the method of claim 94, wherein said symptom is one or more of formation of granulomas, fatigue, weight loss, fever, aches, pains, arthritis, dry eyes, swelling of the knees, blurry vision, shortness of breath, cough and skin lesions.

96. A compound, or a pharmaceutically acceptable salt thereof, for use according to any one of claims 1-5, or a pharmaceutical composition according for use according to claim 75 or 77, or the method according to claim 78 or 79, wherein the compound is administered at a dose of 100 mg b.i.d.

97. A compound, or a pharmaceutically acceptable salt thereof, for use according to any one of claims 1-5, or a pharmaceutical composition according for use according to claim 75 or 77, or the method according to claim 78 or 79, wherein the compound is administered at a dose of 200 mg qd.

98. A compound, or a pharmaceutically acceptable salt thereof, or a pharmaceutical composition or the method according to claim 97, wherein the compound is administered over a period of at least 6 months.

99. A pharmaceutical composition for use in the prophylaxis and/or treatment of giant cell arteritis, comprising the compound according to Formula I, or a pharmaceutically acceptable salt thereof, and a pharmaceutically acceptable carrier, excipient, or diluent.

100. A pharmaceutical composition for use according to claim 99, comprising a further therapeutic agent.

101. The compound or a pharmaceutically acceptable salt thereof, according to claim 5 or the pharmaceutical composition for use according to claim 100, wherein the further therapeutic agent is an agent for the prophylaxis and/or treatment of giant cell arteritis.

102. A compound, or a pharmaceutically acceptable salt thereof, for use according to any one of claims 1-5, or a pharmaceutical composition according to claim 99 or 100 in an individual afflicted with giant cell arteritis.

103. A compound, or a pharmaceutically acceptable salt thereof, for use according to any one of claims 1-5, or a pharmaceutical composition according to claim 99 or 100 in an individual afflicted with giant cell arteritis, as characterized by CRP level greater than 3 mg/L.

104. A compound, or a pharmaceutically acceptable salt thereof, for use according to any one of claims 1-5, or a pharmaceutical composition according to claim 99 or 100 in an individual afflicted with giant cell arteritis, as characterized by a erythrocyte sedimentation rate greater than 30 mm/h.

105. A compound, or a pharmaceutically acceptable salt thereof, or a pharmaceutical composition for use according to any one of claims 1-5, or a pharmaceutical composition according to claim 99 or 100, wherein the compound is administered at a dose of 100 mg b.i.d.

106. A compound, or a pharmaceutically acceptable salt thereof, or a pharmaceutical composition for use according to any one of claims 1-5, or a pharmaceutical composition according to claim 99 or 100, wherein the compound is administered at a dose of 200 mg qd.

107. A compound, or a pharmaceutically acceptable salt thereof, or a pharmaceutical composition for use according to any one of claims 1-5, or a pharmaceutical composition according to claim 99 or 100, wherein the compound is administered over a period of least 4, 6, 8, 10, 12, 14, 16, 20, or 24 weeks.

108. A compound, or a pharmaceutically acceptable salt thereof, or a pharmaceutical composition for use according to any one of claims 1-5, or a pharmaceutical composition according to claim 99 or 100, wherein the compound is administered over a period of least 12 weeks.

109. A method for the treatment the prophylaxis and/or treatment of giant cell arteritis comprising the steps of:
a) measuring the ESR of an individual,
b) measuring the CRP level of an individual,
c) comparing said ESR and/or CRP level with normal values,
d) determining a daily dose of the compound according to Formula I, or a pharmaceutically acceptable salt thereof comprised between 25 mg and 400 mg for administration to said individual.

110. A compound, or a pharmaceutically acceptable salt thereof, for use according to claims 1-5, or a pharmaceutical composition according to claim 99 or 100 in an individual afflicted with giant cell arteritis, said use comprising the steps of:
a) assaying a blood sample,
b) measuring levels of at least one giant cell arteritis-associated biomarker,
c) administering a therapeutically effective amount of the compound, or a pharmaceutically acceptable salt thereof to the individual,
d) measuring the post-treatment level of said giant cell arteritis-associated biomarker and comparing with pre-treatment levels, and
e) adjusting the dosage of the compound, or a pharmaceutically acceptable salt thereof to be administered to the individual, wherein said dosage is increased when a decrease of less than 2% after 12 weeks in levels of said giant cell arteritis-associated biomarker is detected.

111. A compound, or a pharmaceutically acceptable salt thereof, or a pharmaceutical composition for use according to claim 110, wherein the biomarker is IL6.

112. A compound, or a pharmaceutically acceptable salt thereof, or a pharmaceutical composition for use according to claim 110, wherein the biomarker is IL1.

113. A compound, or a pharmaceutically acceptable salt thereof, or a pharmaceutical composition for use according to claim 110, wherein the biomarker is GM-CSF.

114. A compound, or a pharmaceutically acceptable salt thereof, or a pharmaceutical composition for use according to any one of claims 109-113, wherein the biomarker level is decreased by at least 5%, at least 10%, at 15% compared to pre-treatment level.

115. A compound, or a pharmaceutically acceptable salt thereof, or a pharmaceutical composition for use according to any one of claims 109-113, wherein the biomarker levels are decreased by at least 5% after 12 weeks.
